(19) United States
(12) Patent Application Publication
(10) Pub. No.: US 2011/0183860 A1
(43) Pub. Date: Jul. 28, 2011

(54) Protein Aggregation Domains and Methods of Use Thereof

(75) Inventors: Susan Lindquist, Chestnut Hill, MA (US); Peter Tessier, Waltham, MA (US)

(73) Assignee: Whitehead Institute for Biomedical Research, Cambridge, MA (US)

(21) Appl. No.: 12/441,192

(22) PCT Filed: Sep. 13, 2007
(86) PCT No.: PCT/US07/19910

§ 371 Date: Apr. 7, 2009

Related U.S. Application Data
(60) Provisional application No. 60/844,252, filed on Sep. 13, 2006.
(60) Provisional application No. 60/928,401, filed on May 9, 2007.

(51) Int. Cl. C40B 30/04; C40B 40/10; G01N 33/68; C07K 7/08; C07K 14/00; C07K 7/06; C07K 19/00; C12N 9/96; C07K 1/107

(52) U.S. Cl. 506/9; 506/18; 436/501; 530/326; 530/325; 530/324; 530/327; 530/328; 435/188; 530/350; 530/303; 530/394; 530/359; 530/399; 530/382

(57) ABSTRACT

Using the Sup35 prion proteins of two distantly related yeast species, it is established that prion replication is initiated by small elements of primary sequence, which can be identified using arrays of short peptides. Subtle differences in replication elements govern the formation of distinct aggregate conformations (prion strains) and also determine their species-specific seeding activities. A Sup35 chimera that promiscuously forms prions in more than one species does so by virtue of carrying the replication element of each species. Mutations or conditions that cause the chimera to assemble into distinct prion strains favor recognition of distinct replication elements. Therefore, subtle differences in small sequences that constitute prion replication elements encode important determinants of prion propagation and transmission. The protein aggregation domains, methods for identification thereof, and polypeptides and higher order aggregates including the protein interaction domains, as well as arrays including peptides derived from an aggregation-prone polypeptide are provided.

### BACKGROUND

The ability to form β-sheet rich amyloid fibers appears to be a general property of polypeptides¹⁻³. It is increasingly being recognized that amyloid formation is associated not only with disease, but also with diverse biological functions including long-term memory^{4,5}, cell-adhesion⁶, skin pigmentation⁷ and adaptation to environmental stresses^{8,9}. Prions are an unusual class of amyloid-forming proteins that form self-seeding aggregates that are infectious and can be transmitted from cell to cell within or, in some cases, between organisms. The first identified prion was PrP, a GPI-anchored plasma membrane glycoprotein whose conversion to an aggregated, prion form (PrP^{Sc}) is associated with several fatal neurological diseases¹⁰. Since this discovery, several prions in yeast and fungi have been identified. The most well studied yeast prion is Sup35p, a translation termination factor whose aggregation and conversion to its prion state, [PSI⁺], reduces its activity and increases read-through of stop codons¹¹⁻¹⁴. This read-through reveals hidden genetic variation and creates complex new phenotypes in a single step^{8,9,15}. The capacity of Sup35 to switch to the [PSI⁺] state is highly conserved across diverse yeast species¹⁶⁻²¹.

The transmission of prions between different species is usually limited by a species (e.g., transmission) barrier. However, while it is known that certain prions traverse species barriers, detailed molecular mechanisms describing this process remain elusive despite their importance to both human health and the biology of many other amyloid forming proteins. Previous work suggests that poorly defined aspects of prion sequence and aggregate structure control the efficiency of interspecies transmission. The importance of sequence was first demonstrated using transgenic mice expressing both mouse and hamster PrP^{22,23}. Infectious prions from hamsters do not transmit pathogenic phenotypes to wild-type mice but readily transmit such phenotypes to the transgenic animals (see reference 24 for discussion of nonpathogenic prion replication). Further, changing 5 of 16 residues that differ between the species to make mouse PrP more similar to the hamster PrP rendered the mice susceptible to hamster prions²⁵.

Similar transmission barriers apparently governed by sequence have also been found for Sup35, whose molecular architecture consists of an N-terminal prion domain rich in glutamine and asparagines residues (N), a highly-charged middle domain (M) and a C-terminal domain encoding translation termination function (C). Protein aggregates of the N and M domains, denoted NM, from Candida albicans (Ca) are unable to induce [PSI⁺] efficiently in Saccharomyces cerevisiae (Sc) and vice versa^{17,18,26}. However, aggregates from C. albicans can induce the prion state in S. cerevesiae efficiently if the Ca Sup35 allele is present and vice versa^{14,17}. Aggregates of a chimeric protein formed from the first 40 amino acids of the Sc Sup35, residues 49-143 from Ca Sup35 and residues 124-253 from Sc Sup35 induced the prion state efficiently regardless of which endogenous Sup35 variant was present^{17,18,27}.

The importance of aggregate structure on interspecies prion transmission was highlighted by experiments in which two structurally-distinct strains (CJD or vCJD) of human PrP were transmitted to mice either expressing human or mouse PrP³³⁻³⁵. The mice expressing human PrP were more susceptible to CJD prions, while the WT mice were more susceptible to vCJD prions, even though the prion sequences are identical³³⁻³⁵. The Sc/Ca Sup35 chimera also forms multiple structural strains, and certain strains specifically seed one prion relative to the other^{17,27,30}. Mutations in the Sc/Ca chimera have also been identified that favor the formation of specific strains that selectively seed each of the prion domains³⁰. Further, specific strains of Sc and Ca Sup35 have been identified that appear capable of crossing the Sc/Ca Sup35 transmission barrier at a low efficiency²⁷. These and other results³⁶⁻⁴² suggest an intimate relationship between prion strains and transmission barriers, although little is known about the structural details of different aggregate conformations.

### SUMMARY

The present materials and methods involve protein aggregation domains, and elucidate the manner in which the effects of primary sequence and aggregate structure are related to interspecies prion transmission. It has been speculated that the protein sequence influences the efficiency of cross-species prion transmission primarily by dictating the spectrum of allowed aggregate conformations²⁷. This hypothesis suggests that prions will be efficiently transmitted between two species if their primary sequences encode conformations that both prions can readily adopt. Definitive results supporting this hypothesis have proven elusive due to experimental limitations. Evidence is presented herein by investigating the prion properties of Sup35 based on the ability to form biologically relevant, highly infectious amyloid fibers in vitro from different yeast species^{26,43,44}. Herein, results are presented that suggest a simple molecular connection between primary sequence, aggregate structure and transmission barriers. It was found that small sequence elements that initiate prion replication within the prion domains of Sc and Ca Sup35 can be identified using arrays of short peptides. Further, it was found that these replication elements can initiate certain aggregate conformations that retain the seeding specificity for proteins with an identical recognition sequence. A promiscuous chimera capable of crossing the Sup35 transmission barrier was employed to validate the mechanism. Results demonstrate that short peptide portions of yeast prion proteins, lacking the context provided by some or all of the remainder of the full length polypeptide from which they were derived, bind directly to the full length polypeptide and self-assemble to form higher order aggregates, e.g., fibrils. Furthermore, binding of the polypeptide to the peptide and aggregate formation can take place when the peptide is attached to a solid support. In addition, the aggregate, e.g., fibril, can be detached from the solid support and retain its structure.

In one aspect, methods and reagents useful for identifying protein aggregation domains are provided. In one aspect, an amino acid sequence that is derived from an aggregation-prone polypeptide and to which the aggregation-prone polypeptide binds to form a higher ordered aggregate, e.g., an aggregate referred to in the scientific literature by terms such as “amyloid,” “amyloid fibrils,” “fibrils” (also referred to as “fibers”), “prions,” and the like are provided. By “higher ordered” is meant an aggregate of at least 25 polypeptide subunits, and is meant to exclude the many proteins that are known to include polypeptide dimers, tetramers, or other small numbers of polypeptide subunits in an active complex, although the peptides and polypeptides may form such complexes as well. The term “higher-ordered aggregate” also is meant to exclude random agglomerations of denatured proteins that can form in non-physiological conditions. Without limitation, the amino acid sequences provided herein may be SCHAG sequences as that term is used in U.S. Ser. No. 11/004,418 (e.g., The chimeric polypeptide comprises a SCHAG amino acid sequence as one of its polypeptide segments. By “SCHAG amino acid sequence” is meant any amino acid sequence which, when included as part or all of the amino acid sequence of a protein, can cause the protein to coalesce with like proteins into higher ordered aggregates commonly referred to in scientific literature by terms such as “amyloid,” “amyloid fibers,” “amyloid fibrils,” “fibrils,” or “prions.” In this regard, the term SCHAG is an acronym for Self-Coalesces into Higher-ordered Aggregates.). It will be understood than many proteins that will self-coalesce into higher-ordered aggregates can exist in at least two conformational states, only one of which is typically found in the ordered aggregates or fibrils. The term “self-coalesces” refers to the property of the polypeptide such as those described herein or known in the art to form ordered aggregates with polypeptides having an identical amino acid sequence under appropriate conditions and is not intended to imply that the coalescing will naturally occur under every concentration or every set of conditions. The term “higher-ordered aggregate” is used interchangeably herein with the term “aggregate” unless otherwise indicated.

The short polypeptide segments (“aggregation domains”) identified as described herein may be used for any purpose previously contemplated for protein aggregation domains (see, e.g., U.S. Ser. No. 11/004,418 and PCT/US2006/022460). For example, they can be included within as part of a larger amino acid sequence and cause that amino acid sequence to form a higher order aggregate. Thus in one aspect, an amino acid sequence that, when included as part or all of the amino acid sequence of a polypeptide, can cause the polypeptide to coalesce with like polypeptides (e.g., polypeptides identical or similar in sequence and/or containing the same or a similar aggregation domain) into a higher ordered aggregate is provided.

In certain embodiments the polypeptide is not Sup35 or a region thereof at least 40 amino acids long, e.g., the N, M, or NM domain. In some embodiments the polypeptide is not SEQ ID NO: 131 of PCT/US2006/022460. In certain embodiments the peptides are not derived from the foregoing polypeptides.

Provided herein is a collection, or set, including a plurality of peptides, wherein the peptides are portions of a polypeptide that is prone to aggregation under appropriate conditions (an “aggregation-prone”) polypeptide. In one embodiment, the aggregation-prone polypeptide is a yeast or fungal prion protein. In another embodiment, the aggregation-prone polypeptide is a mammalian prion protein. In another embodiment, the aggregation-prone polypeptide is any polypeptide known to self-aggregate in vitro or in vivo. In one embodiment the polypeptide is any polypeptide that forms amyloid. In one embodiment the polypeptide is any polypeptide wherein aggregates formed from the polypeptide and/or from fragments of the polypeptide play a role in disease. Polypeptides and diseases of interest include amyloidβ protein, associated with Alzheimer's disease; immunoglobulin light chain fragments, associated with primary systemic amyloidosis; serum amyloid A fragments, associated with secondary systemic amyloidosis; transthyretin and transthyretin fragments, associated with senile systemic amyloidosis and familial amyloid polyneuropathy I; cystatin C fragments, associated with hereditary cerebral amyloid angiopathy; β2-microglobulin, associated with hemodialysis-related amyloidosis; apolipoprotein A-1 fragments, associated with familial amyloid polyneuropathy II; a 71 amino acid fragment of gelsolin, associated with Finnish hereditary systemic amyloidosis; islet amyloid polypeptide fragments, associated with Type II diabetes; calcitonin fragments, associated with medullary carcinoma of the thyroid; prion protein and fragments thereof, associated with spongiform encephalopathies; atrial natriuretic factor, associated with atrial amyloidosis; lysozyme and lysozyme fragments, associated with hereditary non-neuropathic systemic amyloidosis; insulin, associated with injection-localized amyloidosis; and fibrinogen fragments, associated with hereditary renal amyloidosis. The polypeptide can be a full length polypeptide or a fragment thereof that self-assembles to form an aggregate. The length of the fragment may be, e.g., between 10 amino acids up to the full length of the polypeptide, e.g., at least 10, 20, 50, 100, 200, 300, or 500 amino acids, etc., provided that the fragment contains a domain that mediates self-assembly to form higher ordered aggregates. The fragment may encompass between 20-100% of the total polypeptide sequence, e.g., 30-100%, 40-100%, 50-100%, 60-100%, 70-100%, 80-100%, or 90-100% of the total sequence. The collection may contain, e.g., up to 10, 50, 100, 150, 200, 250, or more different peptides. Collectively, in various embodiments, the peptides may encompass between 20-100% of the total polypeptide sequence, e.g., 30-100%, 40-100%, 50-100%, 60-100%, 70-100%, 80-100%, or 90-100% of the total sequence. The peptides may be, e.g., 6-12, 8-15, 10-20, 10-30, 20-30, 30-40, or 40-50 amino acids in length. In some embodiments, the peptides overlap in sequence by between, e.g., 1-25 residues, e.g., between 5-20 residues, or between 10-15 residues. In some embodiments, the peptides “scan” at least a portion of the polypeptide, i.e., the starting positions of the peptides with respect to the polypeptide are displaced from one another (“staggered”) by X residues where X is, for example, between 1-10 residues or between 1-6 residues or between 1-3 residues. In one embodiment, the starting positions of the peptides with respect to the polypeptide sequence are staggered by 1 amino acid. For example, a first peptide corresponds to amino acids 1-20; a second peptide corresponds to amino acids 2-21; a third peptide corresponds to amino acids 3-22, etc. In another embodiment, the starting positions of the peptides with respect to the polypeptide sequence are staggered by 2 amino acids. For example, a first peptide corresponds to amino acids 1-20; a second peptide corresponds to amino acids 3-22; a third peptide corresponds to amino acids 5-23, etc. The collection need not include the N-terminal or C-terminal amino acid of the polypeptide. The collection could span any N-terminal, C-terminal, or internal portion of the polypeptide. The peptides could include or further include a detectable label, a reactive moiety, a tag, a spacer, a crosslinker, etc. The peptides need not all be the same length and need not all fall within any single range of lengths.

Further, provided herein is an array including a collection of peptides. The term “array” is used herein consistently with its meaning in the art. An array typically includes a surface having a plurality of discrete regions (“features”), each of which typically has a particular physical, chemical or biological characteristic, chemical composition, specific binding ability, etc. An array has multiple features including different moieties (for example, different peptides) such that a feature at a predetermined location (an “address”) on the array is distinguishable from other features. The features may be disposed on the surface in an orderly arrangement, e.g., in a plurality of mutually perpendicular rows and columns, though this need not be the case. In certain embodiments the binding reagents are proteins, e.g., peptides, and the array is a protein array. The surface could be made of any suitable material known in the art, e.g., glass, plastic, metal.

The array may include up to 10, 100, 1000, or more features. The features may be disposed in close proximity to one another on a surface such as a slide, wherein they are not separated into individual wells, or on a membrane or filter. Alternately, the peptides could be provided in individual wells of a microwell plate (e.g., a 96, 384, or 1536 well plate) or any other multiwell article of manufacture. In some embodiments the vessel is microfabricated. Methods for making such arrays are known in the art and include a wide variety of printing techniques (e.g., contact or non-contact printing), automated or manual mechanical deposition, as well as synthesis in situ. See, e.g., U.S. Pat. Nos. 6,630,358; 6,475,809; 6,815,078; 7,067,322. In some embodiments the array is a microengraved array and may fit on a glass slide (1 inch×3 inch). In some embodiments an array of microwells is fabricated by photolithography, e.g., soft lithography of slabs of poly(dimethylsiloxane) or another suitable polymer. The peptides could be covalently or noncovalently attached to the surface. They could be directly attached to the surface or attached via a linker. In some embodiments the surface is modified to contain a binding moiety or reactive moiety that binds to or reacts with the peptide. The concentration and number of peptide molecules in each feature, the feature size, and the distance between features, etc. could vary. The Examples provide some suitable values. For example, the peptide concentration can be between 1 μm and 5 μm. Embodiments include peptide concentration ranges between 0.001 to 1000 times the concentration range provided in the Examples, e.g., between 0.01 to 100 times the concentration range provided in the Examples, between 0.1 to 10 times or between 0.5 to 5 times the concentration range provided in the Examples. In some embodiments the concentration of peptide in the arrayed spots (or attached to any support) is greater than the concentration of the polypeptide in solution. In some embodiments the concentration of peptide in the arrayed spots (or attached to any support) is less than the concentration of the polypeptide in solution. In some embodiments the concentration of peptide in the arrayed spots (or attached to any support) is between 1 and 10,000 times the concentration of the polypeptide in solution, e.g., between 10 and 5,000 the concentration of the polypeptide in solution, between 100 and 1000 times the concentration of the polypeptide in solution, etc. In another embodiment, the peptides are attached to particles which in one embodiment are distinguishable from one another. The particles may be coded by any of a variety of methods. For example, they may incorporate different detectable moieties such as fluorescent dyes; they may include different oligonucleotide or peptide tags that allow their differential detection and/or isolation, etc. In other embodiments, the peptides can be provided in any assay format that allows for multiplexed protein detection and/or measurement.

Methods for identifying an aggregation domain of a polypeptide are provided herein. One such method includes steps of providing an array including a plurality of peptides, wherein the peptides are fragments of a polypeptide that spontaneously aggregates into a higher order structure under appropriate conditions; contacting the array with the polypeptide; and identifying a peptide to which the polypeptide binds, thereby identifying an aggregation domain of the polypeptide. A method of identifying a peptide that seeds self-assembly of an aggregation-prone polypeptide includes: providing an array including a plurality of peptides, wherein the peptides are fragments of a polypeptide that spontaneously aggregates into a higher order structure under appropriate conditions; contacting the array with the polypeptide; and identifying a peptide that induces assembly of the polypeptide to form a higher ordered aggregate, thereby identifying a peptide that seeds self-assembly of the polypeptide. The contacting can take place under a variety of conditions of temperature, pH, osmolarity, salt concentration, etc. In some embodiments the conditions resemble physiological conditions, e.g., conditions under which the polypeptide self-assembles in nature. The Examples provide suitable conditions, but one of skill in the art will appreciate that the conditions could be varied. A suitable pH may be 5-10, e.g., 6-9, e.g., about 7. A suitable temperature may be 20-50° C., e.g., 30-45° C., e.g., 35-40° C., or 37° C. The polypeptide is provided in soluble form. The polypeptide may be present in solution as monomers, dimmers, or oligomers, e.g., including 3-5 individual molecules. In some embodiments the solution includes a mixture of monomers, dimmers, and oligomers. In some embodiments at least 25%, 50%, 75%, or 90% of the polypeptide by weight is present in monomeric form. In some embodiments the polypeptide is denatured prior to contacting with the peptides. The contacting could take place over a time period ranging from 10 minutes to several hours, days, or longer, e.g., between 1 and 24 hours, between 2 and 12 hours, between 24 and 48 hours, etc.

The methods may be applied to any polypeptide that forms higher ordered aggregates. For example, the methods may be applied to identify aggregation domains and/or to investigate the misfolding specificity of polypeptides such as Sup35 proteins, Ure2 proteins, New1 proteins, Rnq1 proteins, mammalian prion proteins, amyloid precursor protein, Aβ40, Aβ42, immunoglobulin (Ig) light chain, serum amyloid A, wild type or variant transthyretin, lysozyme, BnL, cystatin C, β2-microglobulin, apoliprotein A1, gelsolin or a mutant thereof, lactotransferrin, islet amyloid polypeptide, fibrinogen, prolactin, insulin, calcitonin, atrial natriuretic factor, α-synuclein, Huntingtin, superoxide dismutase, or α1-chymotrypsin. In certain embodiments the methodology does not require site-specific labeling. Furthermore, the methods considerably reduce the extent of experimentation needed to identify minimal protein aggregation domains. As such, the methods are applicable to the straightforward identification of protein interaction domains within any aggregation-prone polypeptide. One of skill in the art will readily be able to identify the full length sequences of these or any other aggregation-prone polypeptide of interest by reference to public databases as well as the scientific and patent literature. For example, the sequence of Sc Sup35 is provided in U.S. Ser. No. 11/004,418.

Aggregation domains of the yeast prion proteins Saccharomyces cerevisiae (Sc) Sup35 and Candida albicans (Ca) Sup 35 are identified using the methods provided herein. The methods provided herein were used to identify a variety of peptides located between amino acids 1-40 of Sc Sup35 as capable of binding to full length Sc Sup35 (but not Ca Sup35) to form higher ordered aggregates. Exemplified herein is a peptide that consists of amino acids 10-29 of Sc Sup35. The methods provided herein were also used to identify a variety of peptides including amino acids 69-76 of Ca Sup35 as capable of binding to full length Ca Sup35 (but not to Sc Sup35) to form higher ordered aggregates.

In one embodiment methods of forming a higher ordered aggregate (e.g., a fibril) includes the steps of: (i) providing a peptide including a protein aggregation domain; (ii) contacting the peptide with a longer polypeptide including the aggregation domain; and (iii) maintaining the peptide and the longer polypeptide for a time sufficient for formation of a higher ordered aggregate. The longer polypeptide may be one from which the aggregation domain is derived. Structures made using the method provided herein are also provided. In some embodiments the structure includes a peptide including a protein aggregation domain and a longer polypeptide that also includes the aggregation domain. In some embodiments the structures consist of at least 25%, 50%, 75%, 90%, 95% or more polypeptide by weight.

The compositions of matter and methods provided herein are of use to build structures of a desired shape and composition. Peptides can be deposited on a surface in any desired pattern and combination. The surface is then contacted with a solution containing one or more polypeptide(s) that contain aggregation domain(s) corresponding to one or more of the peptides. The polypeptides assemble to form higher ordered aggregates at the positions where the peptide that induces their self-assembly is located on the surface. In some embodiments the structures are nanostructures. Such structures may have at least one dimension, e.g., height, width, length, less than 1 mm. In some embodiments a conductive or resistive substance, e.g., a suitable metal, polymer, or ceramic material, is deposited on the structure.

The methods provided herein are of use to capture and/or detect a polypeptide. They provide an alternative to polypeptide capture and/or detection mediated by antibodies, aptamers, or other specifically designed binding moieties such as affibodies, etc. The methods also do not require use of cross-linking agents. Thus, methods for assembling polypeptide structures that do not employ cross-linking agents are provided. The structures are, in certain embodiments, highly stable to conditions that would typically cause denaturation or disassembly of multi-subunit proteins. For example, they are in certain embodiments stable to detergents, e.g., 2% SDS, denaturants, elevated temperature, etc. In some embodiments the methods make use of naturally occurring polypeptide fragments that mediate assembly of a corresponding polypeptide, i.e., a polypeptide that includes the fragment or a fragment sufficiently similar to nucleate assembly of the polypeptide to form higher ordered aggregate.

In another aspect, a chimeric polypeptide including a protein aggregation domain described herein and a polypeptide of interest is provided. The protein aggregation domain may be located N-terminal or C-terminal to the polypeptide of interest. By “polypeptide of interest” is meant any polypeptide that is of commercial or practical interest and that includes an amino acid sequence encodable by the codons of the universal genetic code. Exemplary polypeptides of interest include: enzymes that may have utility in chemical, food-processing (e.g., amylases), or other commercial applications; enzymes having utility in biotechnology applications, including DNA and RNA polymerases, endonucleases, exonucleases, peptidases, and other DNA and protein modifying enzymes; polypeptides that are capable of specifically binding to compositions of interest, such as polypeptides that act as intracellular or cell surface receptors for other polypeptides, for steroids, for carbohydrates, or for other biological molecules; polypeptides that include at least one antigen binding domain of an antibody, which are useful for isolating that antibody's antigen; polypeptides that include the ligand binding domain of a ligand binding protein (e.g., the ligand binding domain of a cell surface receptor); metal binding proteins (e.g., ferritin (apoferritin), metallothioneins, and other metalloproteins), which are useful for isolating/purifying metals from a solution containing them for metal recovery or for remediation of the solution; light-harvesting proteins (e.g., proteins used in photosynthesis that bind pigments); proteins that can spectrally alter light (e.g., proteins that absorb light at one wavelength and emit light at another wavelength); regulatory proteins, such as transcription factors and translation factors; and polypeptides of therapeutic value, such as chemokines, cytokines, interleukins, growth factors, interferons, antibiotics, immunopotentiators and immunosuppressors, and angiogenic or anti-angiogenic peptides, marker proteins such as a fluorescing protein (e.g., green fluorescent protein or firefly luciferase), an antibiotic resistance-conferring protein, a protein involved in a nutrient metabolic pathway that has been used in the literature for selective growth on incomplete growth media, or a protein (e.g. β-galactosidase, an alkaline phosphatase, or a horseradish peroxidase) involved in a metabolic or enzymatic pathway of a chromogenic or luminescent substrate that results in the production of a detectable chromophore or light signal that has been used in the literature for identification, selection, or quantitation, proteins (e.g., glutathione S-transferase or Staphylococcal nuclease) that has been used in the literature as a fusion partner for the express purpose of facilitating expression or purification of other proteins. Also provided are nucleic acids that encode any of the peptides or polypeptides disclosed herein. Also provided are expression vectors including any of the nucleic acids that encode a peptide or polypeptide disclosed herein. Also provided are host cells (e.g., bacterial, fungal, insect, mammalian cells) that contain or express any of the nucleic acids that encode a peptide or polypeptide disclosed herein.

Methods for identifying a candidate agent for modulating protein aggregation, e.g., enhancing or inhibiting or altering the kinetics of protein aggregation are provided herein. One such method includes: (i) providing a composition including an aggregation-prone polypeptide, a test agent, and a peptide derived from the aggregation-prone polypeptide, wherein the peptide is capable of binding to the aggregation-prone polypeptide in the absence of the test agent; and (ii) identifying the agent as a candidate agent for modulating protein aggregation if presence of the test agent alters the extent or rate of binding of the peptide and the polypeptide. One such method includes: (i) providing a composition including an aggregation-prone polypeptide, a test agent, and a peptide derived from the aggregation-prone polypeptide, wherein the peptide is capable of seeding aggregation of the aggregation-prone polypeptide in the absence of the test agent; and (ii) identifying the agent as a candidate agent for modulating protein aggregation if presence of the test agent alters the extent or rate of aggregate formation. “Derived from” means that the peptide is a fragment of the polypeptide or is sufficiently similar in sequence to a fragment of the polypeptide to nucleate self-assembly of the polypeptide to form an aggregate.

Methods for identifying a candidate agent for inhibiting protein aggregation are provided herein. One such method includes: (i) providing a composition including an aggregation-prone polypeptide, a test agent, and a peptide derived from the aggregation-prone polypeptide, wherein the peptide is capable of binding to the aggregation-prone polypeptide in the absence of the test agent; and (ii) identifying the agent as a candidate agent for inhibiting protein aggregation if presence of the test agent reduces the binding of the peptide and the polypeptide. The polypeptide may be, e.g., a polypeptide whose aggregation is associated with mammalian disease. One such method includes: (i) providing a composition including an aggregation-prone polypeptide, a test agent, and a peptide derived from the aggregation-prone polypeptide, wherein the peptide is capable of seeding aggregation of the aggregation-prone polypeptide in the absence of the test agent; and (ii) identifying the agent as a candidate agent for inhibiting protein aggregation if presence of the test agent reduces aggregation of the polypeptide. The polypeptide may be, e.g., a polypeptide whose aggregation is associated with mammalian disease. The peptide can be any peptide that binds to an aggregation-prone polypeptide. The peptide can be any peptide that binds to an aggregation-prone polypeptide. In certain embodiments, the peptide is one that forms higher order aggregates when contacted with the aggregation-prone polypeptide. The peptide may be any peptide identified according to the methods for identifying aggregation domains described herein. The peptide may be a fragment of the aggregation-prone polypeptide, or a peptide at least 80%, 90%, 95%, or more identical to such a fragment (e.g., 100% identical to such a fragment). In any of the embodiments, the peptide could be contained within a longer peptide. For example, the peptide that nucleates self-assembly of the polypeptide could be extended at either or both ends. The percent identity between a sequence of interest and a second sequence over a window of evaluation may be computed by aligning the sequences, determining the number of residues (amino acids) within the window of evaluation that are opposite an identical residue (optionally allowing the introduction of gaps to maximize identity), dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. In some embodiments % identity is determined permitting introduction of gaps while in other embodiments not permitting the introduction of gaps. In one embodiment, the peptide is derived from a fragment of the polypeptide by making not more than 1, 2, 3, 4, or 5 additions, deletions, and/or substitutions in any combination. In some embodiments the window of evaluation is the full length of the peptide.

In one embodiment of the method, the polypeptide and the peptide are contacted with one another in the absence of the test agent under conditions suitable for binding and are allowed to bind. The test agent is then added, and its ability to disrupt aggregates is assessed. In one embodiment, the polypeptide and the peptide are contacted with one another in the absence of the test agent under conditions suitable for binding and the test agent is added a short time thereafter, e.g., before substantial binding has occurred. The ability of the test agent to inhibit aggregate formation is assessed. Standard methods of assessing complex formation or disruption can be employed. For example, the aggregates can be imaged and/or detection based on mass or alteration in other physical properties can be used. The polypeptide can be labeled, e.g., with a fluorescent or luminescent moiety to facilitate detection of aggregates. The polypeptide could include an epitope tag to facilitate detection using an enzyme-linked or otherwise detectable antibody that binds to the tag. In certain embodiments, it is not necessary to determine whether the test agent inhibits formation of or disrupts higher order aggregates.

A variety of different candidate agents can be tested. A candidate agent can be any molecule or supramolecular complex, e.g. polypeptides, peptides (which is used herein to refer to a polypeptide consisting of 100 amino acids or less, e.g., 8-60 amino acids), small organic or inorganic molecules (i.e., molecules having a molecular weight less than 1,500 Da, 1000 Da, or 500 Da in size), polysaccharides, polynucleotides, etc. which is to be tested for ability to modulate aggregate formation or disrupt aggregates that have already formed. In some embodiments, the candidate agents are organic molecules, particularly small organic molecules, including functional groups that mediate structural interactions with proteins, e.g., hydrogen bonding, and typically include at least an amine, carbonyl, hydroxyl or carboxyl group, and in some embodiments at least two of the functional chemical groups. The candidate agents may include cyclic carbon or heterocyclic structures and/or aromatic or polyaromatic structures substituted with one or more chemical functional groups and/or heteroatoms. Candidate agents are obtained from a wide variety of sources, as will be appreciated by those in the art, including libraries of synthetic or natural compounds.

In some embodiments, candidate agents are synthetic compounds. Numerous techniques are available for the random and directed synthesis of a wide variety of organic compounds and biomolecules. In some embodiments, the candidate modulators are provided as mixtures of natural compounds in the form of bacterial, fungal, plant and animal extracts, fermentation broths, etc., that are available or readily produced. In some embodiments, a library of compounds is screened. The term “library of compounds” is used consistently with its usage in the art. A library is typically a collection of compounds that can be presented or displayed such that the compounds can be identified in a screening assay. In some embodiments compounds in the library are housed in individual wells (e.g., of microtiter plates), vessels, tubes, etc., to facilitate convenient transfer to individual wells or vessels for contacting cells, performing cell-free assays, etc. The library may be composed of molecules having common structural features which differ in the number or type of group attached to the main structure or may be completely random. Libraries include but are not limited to, for example, phage display libraries, peptide libraries, polysome libraries, aptamer libraries, synthetic small molecule libraries, natural compound libraries, and chemical libraries. Methods for preparing libraries of molecules are well known in the art and many libraries are available from commercial or non-commercial sources. Libraries of interest include synthetic organic combinatorial libraries. Libraries, such as, synthetic small molecule libraries and chemical libraries can include a structurally diverse collection of chemical molecules. Small molecules include organic molecules often having multiple carbon-carbon bonds. The libraries can include cyclic carbon or heterocyclic structure and/or aromatic or polyaromatic structures substituted with one or more functional groups. In some embodiments the small molecule has between 5 and 50 carbon atoms, e.g., between 7 and 30 carbons. In some embodiments the compounds are macrocyclic. Libraries of interest also include peptide libraries, randomized oligonucleotide libraries, and the like. Libraries can be synthesized of peptides and non-peptide synthetic moieties. Such libraries can further be synthesized which contain non-peptide synthetic moieties which are less subject to enzymatic degradation compared to their naturally-occurring counterparts. Small molecule combinatorial libraries may also be generated. A combinatorial library of small organic compounds may include a collection of closely related analogs that differ from each other in one or more points of diversity and are synthesized by organic techniques using multi-step processes. Combinatorial libraries can include a vast number of small organic compounds. In one embodiment, the methods provided herein are used to screen approved drugs. An approved drug includes any compound (which term includes biological molecules such as proteins and nucleic acids) which has been approved for use in humans by the FDA or a similar government agency in another country, for any purpose. This can be a particularly useful class of compounds to screen because it represents a set of compounds which are believed to be safe and, at least in the case of FDA approved drugs, therapeutic for at least one purpose. Thus, there is a high likelihood that these drugs will at least be safe for other purposes. Natural or synthetically produced libraries and compounds are readily modified through conventional chemical, physical and biochemical means. Chemical (including enzymatic) reactions may be done on the moieties to form new substrates or candidate agents which can then be tested using the methods and peptide compositions provided herein. Known pharmacological agents may be subjected to directed or random chemical modifications, including enzymatic modifications, to produce structural analogs.

In some embodiments, candidate agents include peptides, nucleic acids, and chemical moieties. In one embodiment, the candidate modulators are naturally occurring polypeptides or fragments of naturally occurring polypeptides, e.g., from bacterial, fungal, viral, and mammalian sources. In one embodiment, the candidate modulators are nucleic acids of from about 2 to about 50 nucleotides, e.g., about 5 to about 30 or about 8 to about 20 nucleotides in length. In one embodiment, the candidate modulators are peptides of from about 2 to about 60 amino acids, e.g., about 5 to about 30 or about 8 to about 20 amino acids in length. The peptides may be digests of naturally occurring polypeptides or randomly synthesized peptides that may incorporate any amino acid at any position. In one embodiment a synthetic process can generates randomized polypeptides or nucleic acids, to allow the formation of all or most of the possible combinations over the length of the sequence, thus forming a library of randomized candidate bioactive agents. For example, a library of all combinations of amino acids that form a peptide 7 to 20 amino acids in length could be used. In one embodiment, the library is fully randomized, with no sequence preferences, constraints, or constants at any position. In one embodiment, the library is biased, i.e., some positions within the sequence are either held constant, or are selected from a limited number of possibilities. For example, the nucleotides or amino acid residues may be randomized within a defined class, for example, of hydrophobic, hydrophilic, acidic, or basic amino acids, sterically biased (either small or large) residues, towards the creation of cysteines for cross-linking, prolines for turns, serines, threonines, tyrosines or histidines for phosphorylation sites, etc. The peptides could be cyclic or linear.

The candidate agent identified using the methods provided herein may be useful to modulate the phenotype of a yeast or fungal cell that expresses a yeast prion protein. The candidate agent may be useful to modulate the phenotype of a mammalian cell that expresses a mammalian prion protein. In certain embodiments, the candidate agent inhibits formation of a protein aggregate in the cell. In certain embodiments, the candidate agent inhibits formation of a protein aggregate outside of cells but within a living organism. The candidate agent may be useful for treatment or prophylaxis of a condition or disease associated with protein aggregation. The candidate agent may also be used to regulate formation of higher order aggregates in vitro. In certain embodiments the agent is useful to treat a disease associated with protein aggregation. The agent may be given prophylactically, e.g., before an individual has developed symptoms, or after symptoms develop. In some embodiments the agent inhibits additional aggregate formation. In some embodiments aggregates that have already formed are disrupted by the agent.

In another embodiment the peptide arrays are useful to detect presence of bacteria or other pathogenic organisms that produce polypeptides that self-aggregate. Examples of such polypeptides are curli. Curli are the major proteinaceous component of a complex extracellular matrix produced by many bacteria, e.g., many Enterobacteriaceae such as E. coli and Salmonella spp. (Barnhart M M, Chapman M R. Annu Rev Microbiol. 2006; 60:131-47, 2006).

Curli fibers are involved in adhesion to surfaces, cell aggregation, and biofilm formation. Curli also mediate host cell adhesion and invasion, and they are potent inducers of the host inflammatory response. The methods provided herein can be used to identify sequences within curli-forming polypeptides that mediate their assembly. Alternately, sequences already known to have such properties can be used. The peptide sequences are deposited on a surface. Since curli polypeptides are secreted, they are accessible and able to self-assemble when the bacteria come in contact with the peptide.

The methods provided herein can be used to screen for agents that inhibit biofilm formation or that disrupt biofilms that have already formed. Such agents could be used as components of washes or disinfectant solutions (e.g., in combination with a suitable carrier such as water), to impregnate cleaning supplies such as sponges, wipes, or cloths, or as components of surface coatings (e.g., in combination with a suitable carrier such as a polymeric material) for a variety of medical devices. They could also be used as therapeutic agents in individuals who are susceptible to infection, infected, and/or have an indwelling or implantable device. In some embodiments, the agent is used as a component of a coating for a catheter, stent, valve, pacemaker, conduit, cannula, appliance, scaffold, central line, pessary, tube, drain, trochar or plug, implant, a rod, a screw, or orthopedic appliance. In another embodiment, the agent is used as a component of a coating for a conduit, pipe lining, a reactor, filter, vessel, or equipment which comes into contact with a beverage or food, e.g., intended for human or animal consumption or water or other fluid intended for consumption, cleaning, agricultural, industrial, or other use.

A surface having a peptide that nucleates polypeptide aggregation attached thereto can serve as a sensor for the presence of bacteria. Large volumes of fluid could be efficiently tested, e.g., for water quality control applications, etc. Peptides that specifically mediate self-assembly of polypeptides from different bacteria could be deposited on a surface. The surface is placed in a fluid or medium that is to be tested. The peptide “concentrates” the bacteria by facilitating self-assembly. Following a suitable time period the surface is “stamped” onto culture plates. Growth at a specific position on the plate is correlated with the sequence of the peptide located at a particular position on the surface, thereby identifying the bacteria. Alternately standard bacterial identification methods can be used.

In another embodiment a surface having a peptide that nucleates polypeptide aggregation attached thereto is used to purify a solution. The solution may be, e.g., water or a body fluid. The fluid is contacted with the surface under conditions suitable for self-assembly. After a suitable period of time polypeptides from the solution aggregate on the surface and can thus be efficiently removed. In one embodiment, such a method is used to treat a subject either ex vivo or in vivo. The polypeptides may be attached to beads that are administered to the subject. The beads may be magnetic. In one embodiment the method is used to remove polypeptides from a body fluid in a subject undergoing dialysis. The methods may be used to concentrate any polypeptide that includes a domain that mediates self-aggregation.

Any of the peptides, polypeptides, nucleic acids, aggregates, etc., disclosed herein may be “isolated.” “Isolated” should be understood to mean that the material referred to is separated from one or more substances with which it exists in nature (e.g., is separated from cellular material, separated from other polypeptides, separated from its natural sequence context), is otherwise removed from its natural environment, and/or is produced by a process that involves the hand of man such as recombinant DNA technology, chemical synthesis, etc. An isolated entity may have undergone a single purification step or multiple purification steps.

In one aspect, a peptide comprising at least 15 contiguous amino acids located between amino acids 1-40 of Sc Sup35 is provided. The contiguous amino acids include amino acids 18-22 of Sc Sup35 and assemble with full length Sc Sup35 to form a higher ordered aggregate. The sequence of the polypeptide does not contain more than 50 contiguous amino acids of the sequence of Sc Sup35 outside the region between amino acids 1-40 of Sc Sup35.

In another aspect, a peptide comprising at least 15 contiguous amino acids located between amino acids 59-86 of Ca Sup35 is provided. The contiguous amino acids include amino acids 69-76 of Ca Sup35 and assemble with full length Ca Sup35 to form a higher ordered aggregate. The sequence of the polypeptide does not contain more than 50 contiguous amino acids of the sequence of Ca Sup35 outside the region between amino acids 59-86 of Sc Sup35.

In still another aspect, an array comprising a plurality of peptides is provided. The peptides are fragments of a polypeptide, and the polypeptide is a polypeptide that misfolds or spontaneously aggregates into a higher order structure under appropriate conditions.

In yet another aspect a collection comprising at least 10 different peptides is provided. The peptides are fragments of a polypeptide, wherein the polypeptide is a polypeptide that misfolds or spontaneously aggregates into a higher order structure under appropriate conditions.

In still yet another aspect, the method of forming a higher ordered aggregate includes the steps of: providing a composition comprising (a) a peptide comprising a protein aggregation domain and a polypeptide comprising the protein aggregation domain; and maintaining the composition for a time sufficient for formation of a higher ordered aggregate.

In still another aspect, a method of identifying an aggregation domain of a polypeptide is provided. The methods includes providing an array comprising a plurality of peptides, wherein the peptides are fragments of a polypeptide that spontaneously aggregates into a higher order structure under appropriate conditions. The method also includes contacting the array with the polypeptide and identifying a peptide to which the polypeptide binds, thereby identifying an aggregation domain of the polypeptide.

In other examples, any of the aspects above, or any apparatus or method or composition of matter described herein, can include one or more of the following features.

In various embodiments, the peptide does not contain more than 20 contiguous amino acids of the sequence of Sc Sup35 outside the region between amino acids 1-40 of Sc Sup35. The peptide can be located between amino acids 8-40 of Sc Sup35. The peptide can be located between amino acids 8-32 of Sc Sup35. The amino acid sequence can include amino acids 10-29 of Sc Sup35. The amino acid sequence can include amino acids 11-30 of Sc Sup35.

In some embodiments, the polypeptide is between 10 and 50 amino acids in length. The peptide can be between 15 and 50 amino acids in length. The peptide can be between 15 and 30 amino acids in length. A peptide can be at least 90% identical to any of the peptides described herein. A peptide can have a sequence that differs by not more than 2 amino acid insertions, deletions, or substitutions from that of any of the peptides described herein. A polypeptide can have an amino acid sequence including a first portion that includes any of the peptides described herein. A peptide can include a second portion, wherein the second portion has a biological or chemical activity of interest or includes a detectable, selectable, or reactive moiety.

In certain embodiments, a higher order aggregate including any of the peptides described herein is provided. The higher order aggregate can be a fibril.

In various embodiments, the peptide is attached to a solid support. The peptide can be noncovalently attached to the solid support. The peptide or the higher ordered aggregate can be removed from the solid support.

In some embodiments, the peptides scan across between 20% and 100% of the polypeptide and the N-terminal amino acids of the peptides are located between 1 and 10 amino acids from each other within the polypeptide sequence.

In certain embodiments, the polypeptide can be one whose misfolding or aggregation is implicated in mammalian disease. The peptides can be derived from a polypeptide selected from the group consisting of: Sup35 proteins, Ure2 proteins, New1 proteins, Rnq1 proteins, mammalian prion proteins, amyloid precursor protein, Aβ40, Aβ42, immunoglobulin (Ig) light chain, serum amyloid A, wild type or variant transthyretin, lysozyme, BnL, cystatin C, β2-microglobulin, apoliprotein A1, gelsolin mutants, lactotransferrin, islet amyloid polypeptide, fibrinogen, prolactin, insulin, calcitonin, atrial natriuretic factor, α-synuclein, Huntingtin, superoxide dismutase, and α1-chymotrypsin.

### BRIEF DESCRIPTION OF THE DRAWINGS

FIGS. 1a-1g are graphs showing the results of peptide array analysis of interactions sites within the prion domains of Sc and Ca Sup35. Sc NM was labeled with ALEXA FLUOR® 555 (1 μM, 5% of protein labeled) and incubated with hydrogel-coated glass slides displaying overlapping 20mer peptides derived from its sequence for (a) 2 hrs, (b) 1 day and (c) 1 day after preincubation with unlabeled Sc NM for 5 days. (d)-(g) Quantification of the average fluorescent signal for peptides from Sc and Ca NM after 2 hrs of exposure of either Sc (upper box) or Ca (lower box) NM. FIG. 1h is a photographic image of a peptide array that displays overlapping, 20mer peptides spanning both prion domains that has been co-incubated with Sc (upper box) and Ca (lower box) NM. Each 20mer peptide is displayed at its central residue on the x-axis.

FIGS. 2a-2d are graphs showing the results of functional analysis of the Sc and Ca Sup35 interaction sites by arginine mutagenesis. (a) and (b) The inverse initial seeding rates for a series of single arginine mutants spanning Sc and Ca NM, as well as the average fluorescent signal for both WT prion domains interacting with their own peptides after 2 hrs of exposure. The seeding rates, reported as the ratio of the rates that WT NM (4 μM) relative to mutant NM (4 μM) are seeded by 5% WT NM fibers, were measured using both Thioflavin T fluorescence and SDS resistance. (c) and (d) Effect of the arginine mutations (red), Sc S17R and Ca Y75R, on the association of NM with peptides in both the Sc and Ca interaction sites relative to WT NM (gray). The arginine mutant and WT NM proteins (1 μM, 5% Alexa 647 label) were incubated with the peptide arrays for 2 and 22 hrs for Sc and Ca NM, respectively.

FIGS. 3a-3f are graphs showing the results of analysis of prion transmission barriers generated by mutating the Ca Sup35 interaction site. Peptide array results of (a) Ca WT, (b) Ca Y75A, (c) Ca Y75P and (d) Sc WT NM interacting with peptides in the Ca interaction site. Each peptide array was incubated with 5 μM NM labeled with 20% ALEXA FLUOR® 647 for 20 hrs. (e) Initial seeding rates of each of the prion domains (4 μM) analyzed in (a)-(d) when seeded by WT Ca NM fibers. (f) Frequency of conversion to the prion state when Ca NM-YFP is overexpressed using a GAL 2μ plasmid for 16 hrs when the endogenous prion domain in S. cerevesiae has been replaced with Ca WT and mutant prion domains.

FIGS. 4a-4g are graphs showing the results of analysis of the conformational preference of a Sc/Ca Sup35 chimera. Peptide array results for (a) WT Sc NM at 25° C., (b) WT Ca NM at 25° C., (c) WT Sc/Ca chimera at 25° C., (d) G70, 71, 80, 81A Sc/Ca chimera at 25° C., (e) S17R Sc/Ca chimera at 25° C., (f) WT Sc/Ca chimera at 4° C., and (g) WT Sc/Ca chimera at 37° C. Each prion domain (1 μM, protein labeled with either 5% ALEXA FLUOR® 555 or 647) was incubated with a peptide array for 2 hrs.

FIG. 5 is a graph showing the results of an analysis of Ca NM residues in self-contact by pyrene excimer fluorescence. Single cysteine Ca NM mutants (2.5 μM) were labeled with pyrene (50% label) and seeded into fibers (5% wt/wt). The excimer ratio is reported at the ratio of fluorescence at 465 nm relative to 390 nm when excited at 338 nm.

FIG. 6 is a photograph showing the results of an aTEM image of Sc NM amyloid fibers formed on peptide arrays. Sc NM (2.5 μM, 5% ALEXA FLUOR® 555) was incubated with the peptide array for 2.5 days, washed with 2% SDS and dried. Deposited protein was removed from the slide with a syringe needle, resuspended in water and sonicated. The fibers were then deposited on nickel-coated carbon grids, negatively stained and imaged by TEM. The scale bar is 50 nm.

FIGS. 7a-7f are graphs showing the results of mutational analysis of Sup35 interaction sites. The relative affinity of (a) WT Sc NM for WT Sc peptides, (b) S17R Sc NM for WT Sc peptides and (c) WT Sc NM for Sc peptides containing the mutation S17R. The relative affinity of (a) WT Sc/Ca chimera for WT Ca peptides, (b) G70, 71, 80, 81A Sc/Ca chimera for WT Ca peptides and (c) WT Sc/Ca chimera for Ca peptides containing the mutations G70, 71, 80, 81A. The concentrations and label ratios for all Sc and Sc/Ca chimera NM proteins were 1 μM and 5%, respectively. The Sc and Sc/Ca chimera proteins were incubated with the peptide arrays for 2 and 22 hrs, respectively.

FIGS. 8a-8b are photographs showing the results of Western blot analysis of the expression level of WT and mutant Sup35. The prion domain of the endogeneous copy of Sc Sup35p was replaced with the WT and mutant Ca NM prion domains. Sc Sup35p is approximately 3 kD smaller than the Ca Sup35p proteins.

FIG. 9 is a graph showing the results of peptide array and pyrene excimer studies, demonstrating the intermolecular contacts that overlap with two interaction sites in the Sc NM peptide.

FIG. 10 is a graph showing the results of peptide array and pyrene excimer studies, demonstrating the intermolecular contacts that overlap with two interaction sites in the Ca NM peptide.

FIG. 11 is a schematic illustration of an exemplary peptide analysis method described herein.

FIGS. 12a-12b are illustrations of the Identification of recognition sequences within ScNM using peptide arrays. (See also FIGS. 1a-1g.) FIG. 12a shows an image of a triplicate array of sequential overlapping 20-mer ScNM peptides after incubation with labelled full-length ScNM. FIG. 12b shows the quantification of the fluorescence of labelled full-length ScNM bound to a similar peptide array after two and a half days (5 mM, 75% ALEXA FLUOR® 555). The relative fluorescence intensity (RFU) for each 20-mer peptide is displayed at its central residue on the X-axis.

FIG. 13 is an analysis of CaNM recognition sequences and the species barrier between Sc/Ca NM. (See also FIGS. 1a-1g.) In Particular, FIG. 13 shows the quantification of the fluorescence of labelled full length Sc/Ca NM chimaera (1 mM NM, 5% ALEXA FLUOR® 647) bound to both ScNM and CaNM peptides after two hours of incubation. All fluorescence values are reported as median+s.d.

FIGS. 14a-14d are an analysis of the conformational preference of the Sc/Ca NM chimaera. 14a-14d, Quantification of the relative binding of various labelled full-length NM chimeric proteins to overlapping 20-mer ScNM and CaNM peptides: 14a, Sc/Ca chimaera at 37 uC; 14b, Sc/Ca chimaera at 4 uC; 14c, S17R Sc/Ca chimaera at 25 uC; and 14d, G70, 71, 80, 81A Sc/Ca chimaera at 25 uC. The peptide arrays were incubated with each prion domain (1 mM NM, 5% ALEXA FLUOR 647) for two hours. All fluorescence values are reported as median+s.d.

FIGS. 15a-15b are an analysis of the mutational disruption of the ScNM and CaNM recognition elements. 15a, Quantification of the relative affinity of the full length Sc/Ca NM chimaera for wild-type ScNM peptides (dark bars) and ScNM peptides containing the S17R mutation (light bars). 15b, Quantification of the relative affinity of the full-length Sc/Ca NM chimaera for wild-type CaNM peptides (dark bars) and CaNM peptides containing the G70, 71, 80, 81A mutations (light bars). The peptide arrays were incubated with the NM chimaera (1 mM NM, 5% ALEXA FLUOR® 647) for two hours. All fluorescence values are reported as median+s.d.

FIGS. 16a-16c are diagrams showing the molecular architectures and amino-acid sequences of three Sup35 constructs.

### DETAILED DESCRIPTION

Identification and Analysis of Intermolecular Contacts in Ca NM Fibers

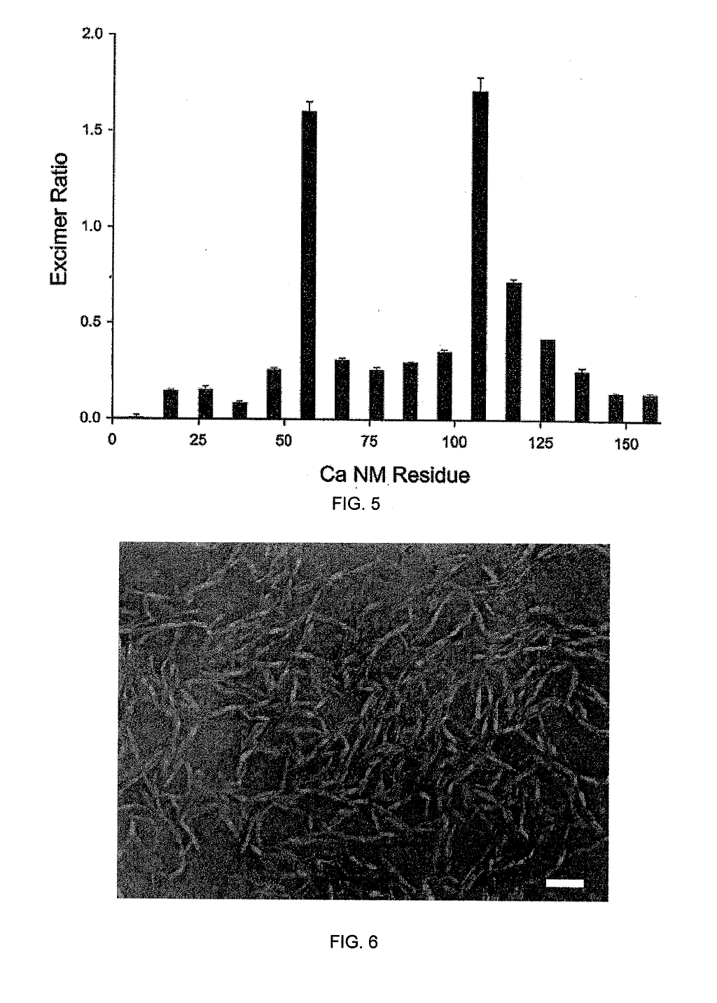

To study whether Ca NM contains discrete sequence elements that govern its seeding and nucleation, residues in self-contact within Ca NM fibers were identified using the fluorophore pyrene. Pyrene has an unusually long fluorescence lifetime (>100 ns) and gives rise to red-shifted excimer fluorescence when two pyrene molecules are in extremely close proximity (4-10 Å). Since wild-type (WT) Ca NM lacks cysteine residues, 15 single cysteine mutants spaced every 10 residues were generated that spanned the N and some of the M domain. The cysteine mutants were labeled with pyrene maleimide, assembled the labeled protein into fibers and measured the level of excimer fluorescence (FIG. 5). Two residues, Q55 and N105, showed strong fluorescence relative to other residues.

The functional significance of Ca Q55 and N105 was tested by measuring the seeding and nucleation efficiency of five arginine mutants (Ca A45R, Q55R, Q65R, N105R and Y115R) located at or near them. It was assumed that replacing residues within or near an intermolecular contact with a charged, bulky arginine residue should inhibit seeding and nucleation. The seeding rates for the arginine mutants relative to WT Ca NM ranged from 0.5 to 1.0, and the lag times for unseeded reactions for the mutants relative to WT Ca NM were 0.9-1.4. The effects of arginine mutations on both Ca NM seeding and nucleation were small relative to the effects of similar mutations at residues found to be in self-contact within Sc NM fibers (minimum initial seeding rate for mutant/WT<0.1) and nucleation (maximum lag time for mutant/WT>2). These results suggested that if discrete regions within Ca NM affect seeding and nucleation significantly, they were not readily identified by pyrene excimer fluorescence in these experiments.

Identification and Analysis of Sup35 Interaction Sites Using Peptide Arrays

A different method for analyzing the functional properties of amyloids was sought. Since the number of residues in or near the intermolecular contact that controls Sc NM nucleation and polymerization in the context of a larger polypeptide is relatively small (−30 residues)⁴⁵, it was hypothesized that short peptides could be used to identify important functional domains within the Sc and Ca NM sequences. Because short peptides from aggregation-prone proteins are often insoluble, denatured peptides were arrayed on reactive glass slides and studied their interaction with soluble, fluorescently labeled NM. Extensive libraries of overlapping peptides (136 peptides for Sc NM, 128 peptides for Ca NM), scanned at intervals of 1-6 residues, were synthesized with 20 residues at their C-terminus derived from each prion domain, a PEG spacer and an N-terminal, double lysine tag for covalent immobilization.

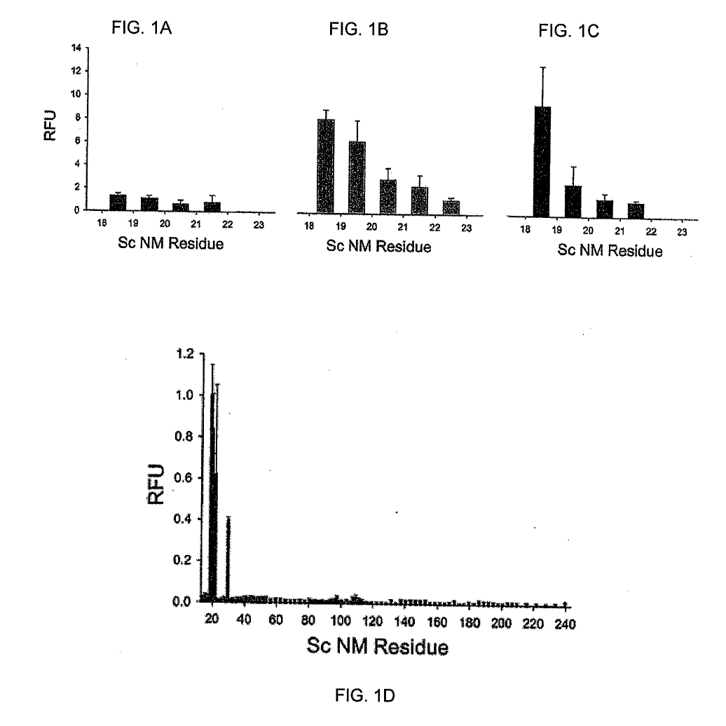

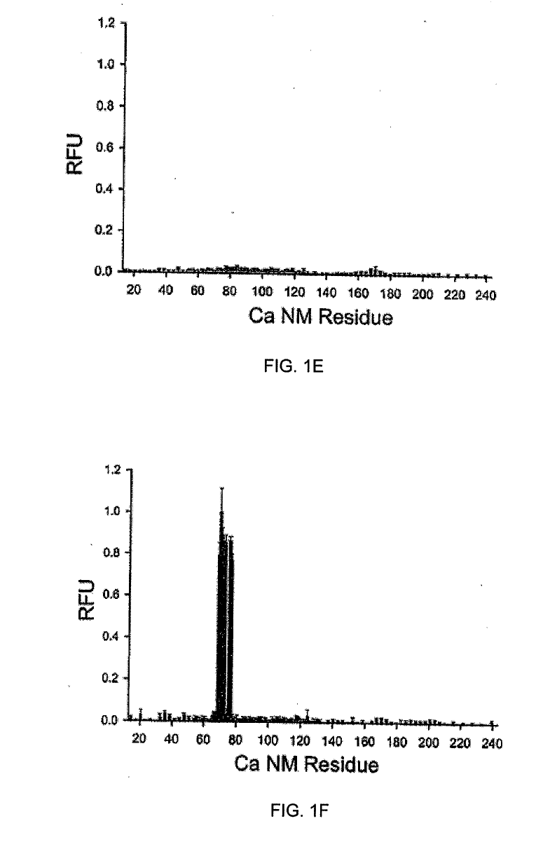

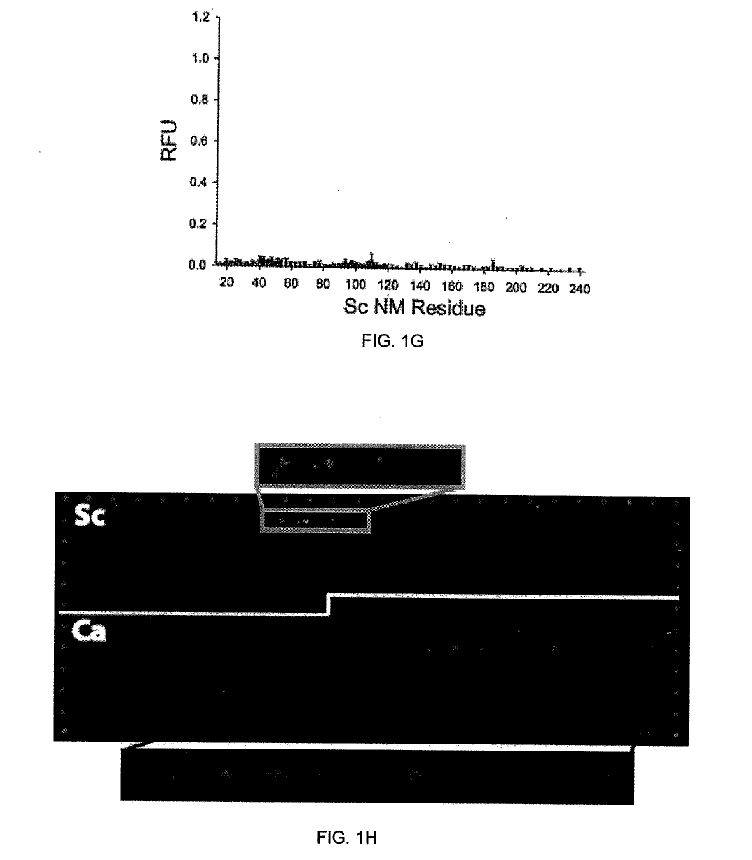

Whether important sequence elements governing Sc Sup35 replication within residues 10-40 of the N domain (residues 10-40) could be identified, which have been studied in the context of Sc Sup35 polypeptides and chimeric Sup35 proteins including portions of Sc and Ca Sup35 polypeptides^{45,46} was studied. Indeed, it was found that incubating labeled Sc NM with peptide arrays for 2 hrs led to a strong interaction with peptides centered at residues 18-22 and these peptides spanned residues 8-32, which was denoted the Sc interaction site (FIG. 1a). Longer incubations (1 day) of labeled NM with the peptide arrays showed that the fluorescence continued to increase (FIG. 1a, gray). Interestingly, the overall signal was not saturable since incubating the arrays for 5 days with unlabeled protein and then adding labeled protein for 1 day resulted in similar fluorescence signals to experiments without pretreatment of unlabeled protein (FIG. 1a). It was suggested that monomeric or oligomeric Sc NM initially interacted with these peptides and then was able to self-interact and assemble on the surface of the arrays, enabling continued protein deposition. Indeed, TEM images of peptide-bound Sc NM confirmed that it was assembled into amyloid fibers (FIG. 1b). It is unlikely that fibers formed in solution and subsequently bound to the peptide arrays since incubation of Sc NM amyloid fibers with the peptide arrays produced less signal than for monomeric protein. Finally, it was confirmed that Sc NM peptide 10-29 could accelerate nucleation of Sc NM fibers by performing unseeded assembly reactions in microtiter plates containing covalently immobilized peptides; the lag time for fiber formation was reduced by 37±1% and 35±1% for wells coated with Sc NM peptide 10-29 relative to wells that were bare or coated with a Sc NM peptide (160-179) located outside the prion domain, respectively.

It was next investigated whether other interaction sites exist in Sc NM by investigating its interaction with peptides spanning Sc NM. Interestingly, incubation of labeled Sc NM for 2 hrs with a peptide array did not produce a significant interaction with peptides containing residues other than 8-40 (FIG. 1d). However, longer incubations (2.5 days) at higher Sc NM concentrations (5 μM, 75% of protein labeled) revealed the presence of a second interaction site spanning residues ˜90-120 (data not shown), which overlaps with the second intermolecular contact identified previously by pyrene excimer fluorescence (86-106)⁴⁵. It was also wondered if Sc NM would interact specifically with its own peptides relative those derived from Ca NM, even though their N domains share a considerable degree of sequence similarity (˜40%) and display similar overrepresentation of four polar, uncharged amino acids (29% vs. 38% Q, 16 vs. 14% N, 16 vs. 13% Y and 17 vs. 14% G for Sc vs. Ca NM). Remarkably, Sc NM shows little affinity for 20mer peptides derived from the Ca NM sequence (FIG. 1e). Quantification of the interaction specificity revealed that the maximal interaction of Sc NM for Sc NM peptides relative to Ca NM peptides is 31 times greater.

Similar interaction sites in Ca NM were also identified. Incubation of labeled Ca NM with a peptide array for 2 hrs revealed that it interacted with a small cluster of peptides centered on residues 69-76 and that spanned residues ˜59-86 (FIG. 1f; SEQ ID NO: 134-263), which are denoted as the Ca interaction site. Notably, this interaction site was in between the residues identified to be in self-contact by excimer fluorescence (Q55 and N105). Longer incubations (2.5 days) at higher Ca NM concentrations (5 μM, 75% labeled protein) revealed a second interaction site that spanned residues 110-130 (data not shown), which is close to the second intermolecular contact in Ca NM fibers identified by excimer fluorescence (105-115). To test the specificity of these interactions the interaction of Ca NM with peptides derived from the Sc NM sequence was examined and little association was observed (FIG. 1g). Quantification of this specificity revealed that the maximal interaction of Ca NM for its own peptides relative to those of Sc NM was 28 times greater. A representative image of a peptide array that was co-incubated with Sc and Ca NM is shown in FIG. 1h. Importantly, the remarkable specificity of both Sup35 variants for interacting with peptides derived from their own sequences correlates well with the significant transmission barrier between these prions observed both in vitro and in vivo^{17,18}.

Functional Analysis of Sup35 Interaction Sites

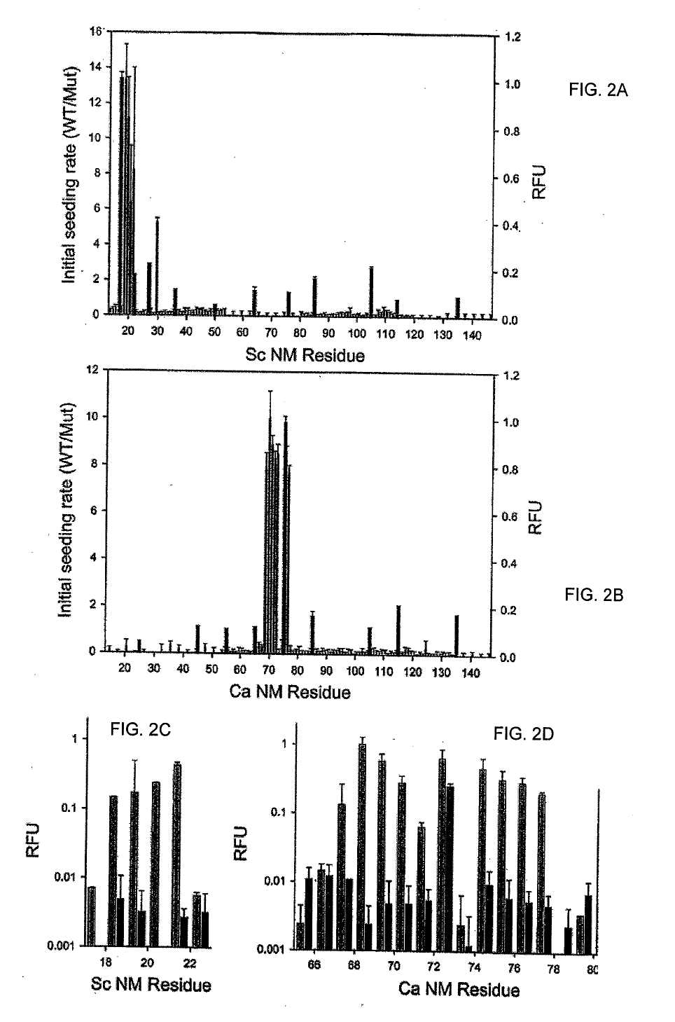

In order to test the importance of the Ca NM interaction site identified by peptide array analysis in seeding and nucleation, as well as to better quantify the effects of the arginine mutants on the conformational conversion of Sc NM, series of 10 single arginine mutants spanning each prion domain was generated to test their effect on seeding and nucleation. The rate at which 5% WT Sc NM fibers seeded Sc WT versus mutant NM (FIG. 2a) was measured, which would correspond to a large ratio if the mutation disrupts the seeding process significantly. It was found that residue S17 is the most sensitive to arginine mutagenesis, which is consistent with a previous report⁴⁶, and is extremely close to the reactive Sc NM peptides centered at residues 18-22. Similar analysis using 5% WT Ca NM fibers to seed WT Ca NM relative to mutant NM revealed that residue Y75 was the most sensitive to mutation (FIG. 2b), which overlaps with the small set of Ca reactive peptides that are centered at residues 69-76. Unseeded assembly reactions for S17R and Y75R were also performed, and found that these mutants increased the lag time relative to the WT prion domains by 2.09±0.06 and 2.85±0.44, respectively. Although the nucleation results were more variable than the seeding results, it was consistently observed that the lag times for Sc S17R and Ca Y75R NM were larger than for the other Sc and Ca NM arginine mutants, respectively (some data not shown).

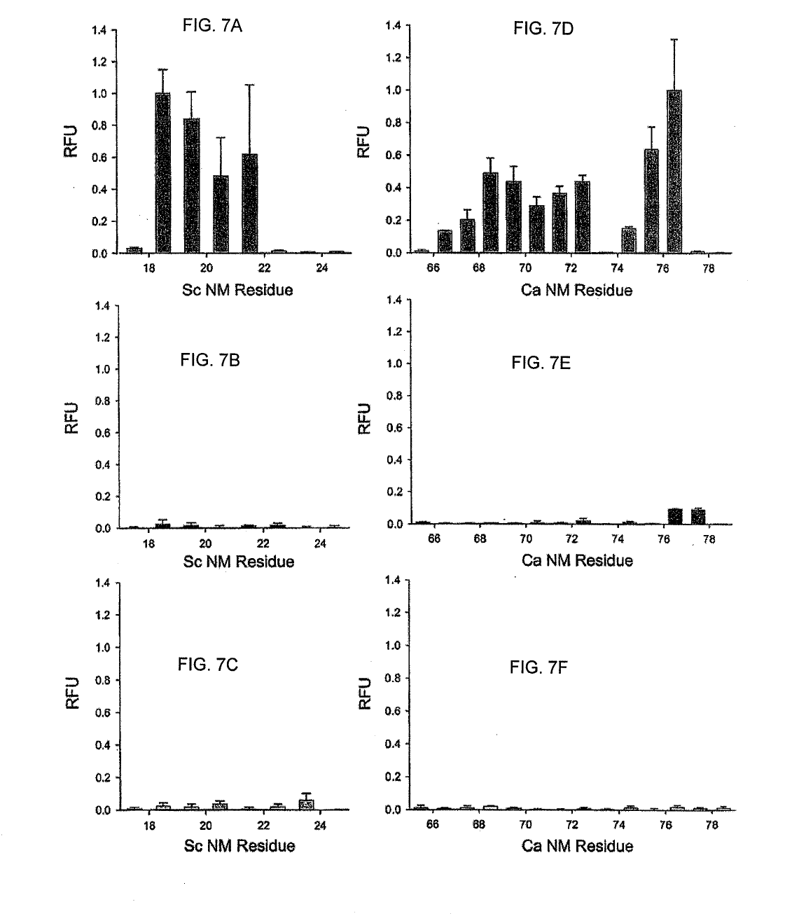

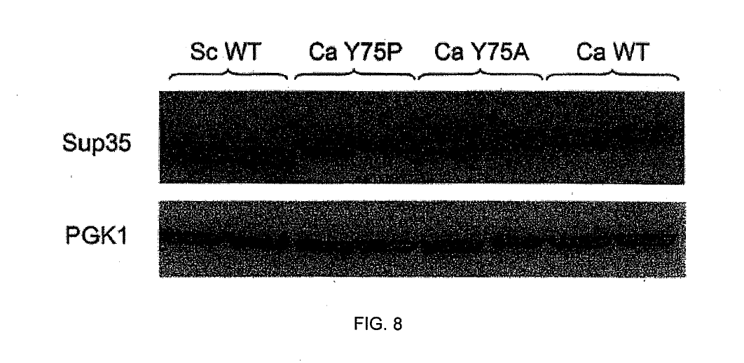

The relative affinity of the fluorescently-labeled arginine mutants, Sc S17R and Ca Y75R NM, for peptides in both interaction sites to investigate how these mutations reduce the seeding and nucleation efficiency of the corresponding prion domains, was also measured. It was found that the association of the arginine mutants with each interaction site is reduced markedly relative to the WT prion domains (FIGS. 2c and 2d), suggesting that they affect the prion assembly by interfering with the association of residues in each corresponding interaction site. Finally, it was asked if these mutants modify intermolecular interactions by disrupting protein-protein interactions or intramolecular interactions by inducing local structure and preventing exposure of the interaction site. The interaction of WT Sc NM and a chimera protein containing most of the N domain of Ca NM^{17,18} with peptides in both interaction sites that contain these mutations or closely related variants was studied. It was found that the interaction of these or similar mutants with each interaction site is reduced significantly regardless of whether the mutations are located in the protein or peptides (FIG. 7), suggesting that these mutations affect intermolecular interactions directly.

Mutational Control of Prion Transmission Barriers

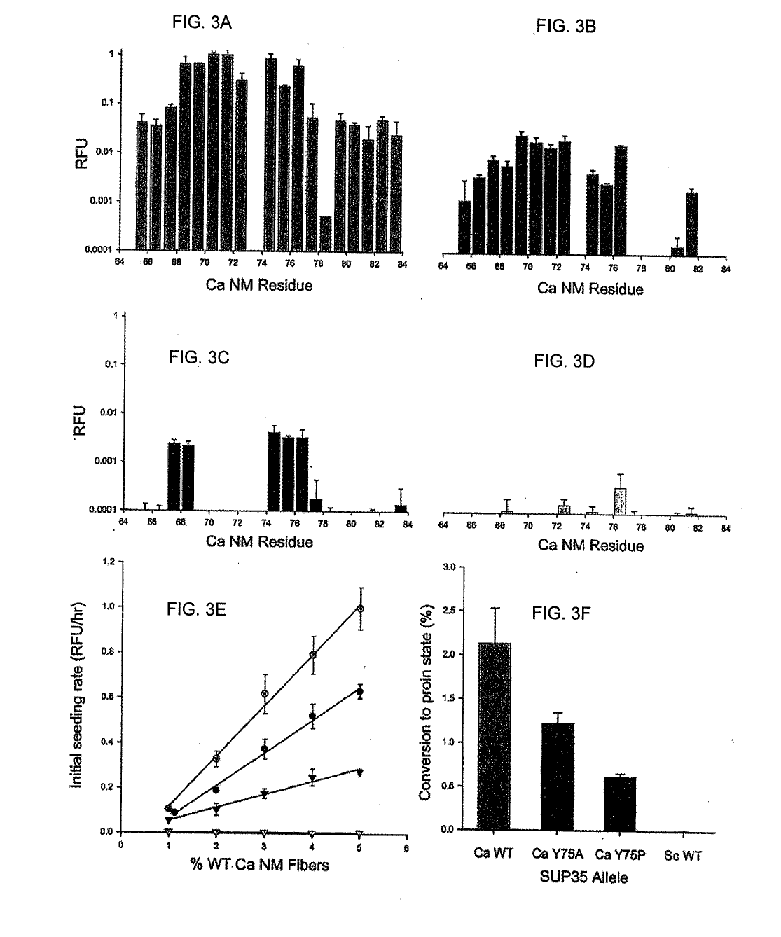

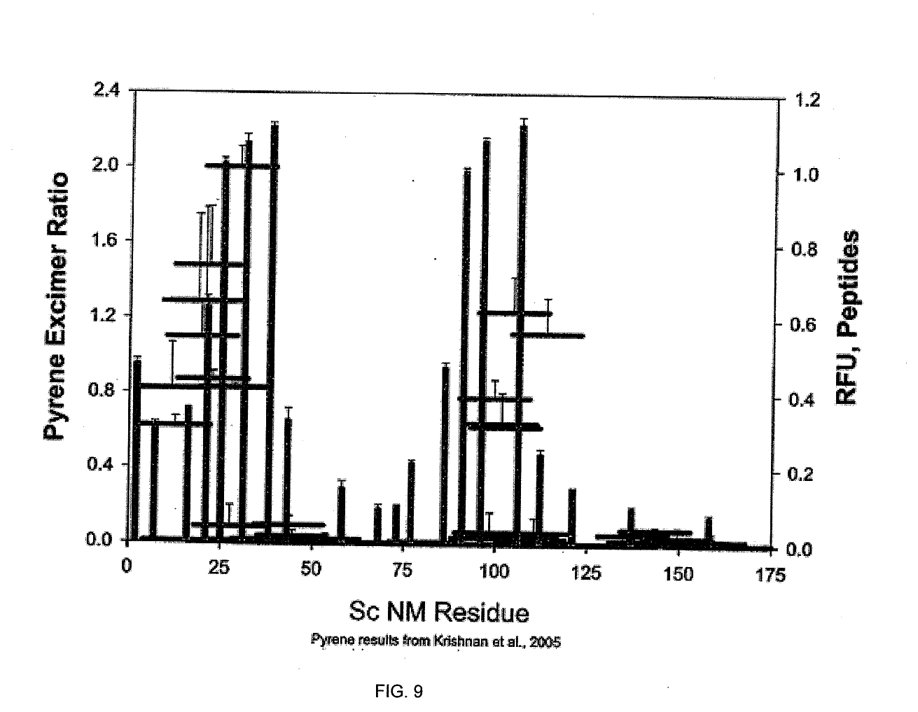

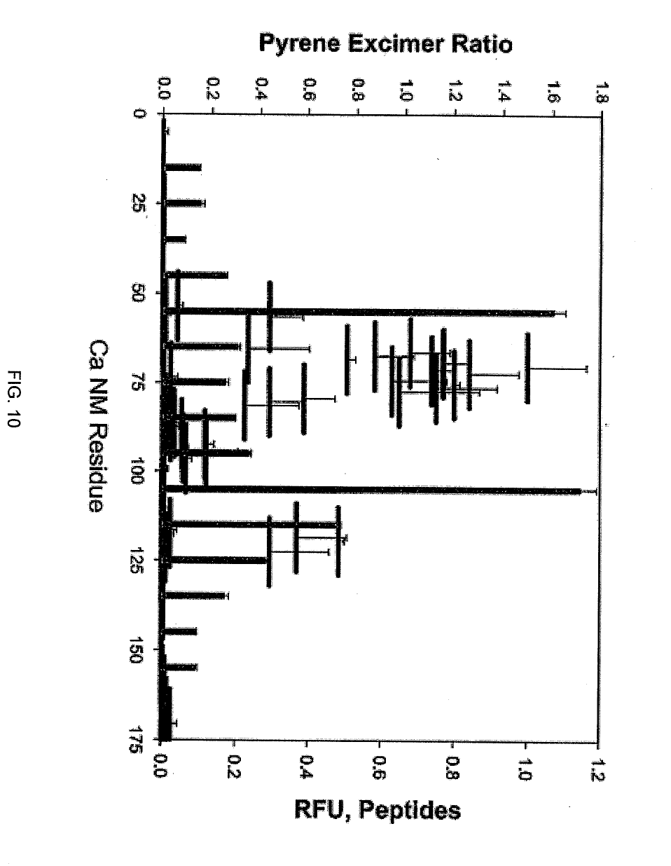

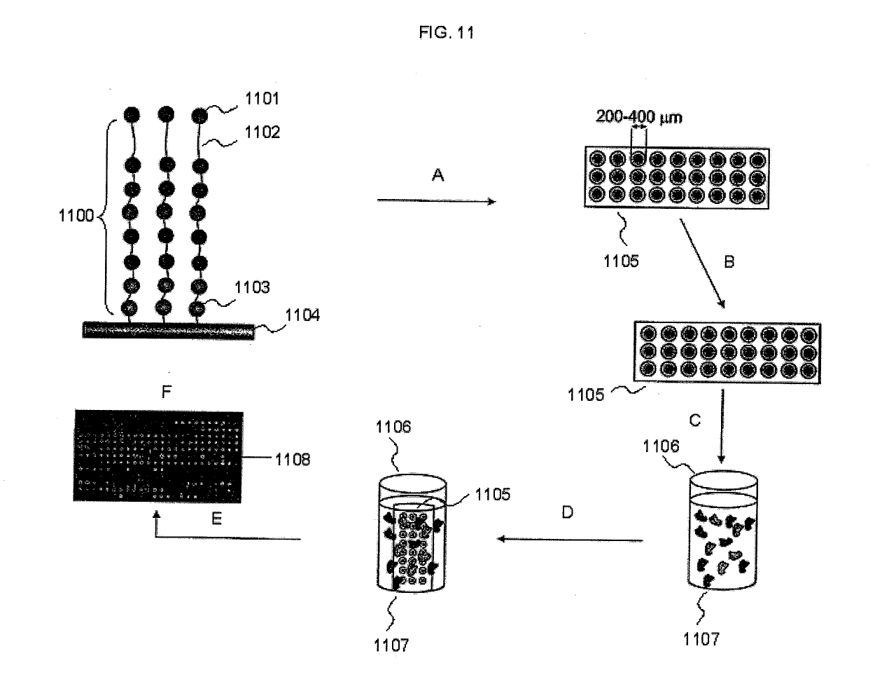

Whether the Sup35 interactions sites that were identified by arginine mutagenesis and peptide array analysis govern prion transmission barriers both in vitro and in vivo was studied. The Ca interaction site was focused upon since little is known about this prion. Two single (Y75A and Y75P) mutants in the Ca interaction site were generated, and their effect on the association of Ca NM with peptides in the Ca interaction site were investigated (FIG. 3a-d). It was found that the interaction of Ca Y75A with peptides in the Ca interaction site was significantly lower than for WT Ca NM, emphasizing the sensitivity of the Ca interaction site to small changes in sequence. Further, Ca Y75P caused an even more significant decrease in binding, but not as great as the WT Sc NM, to peptides in the Ca interaction site. The initial rates that WT Ca fibers seeded WT or mutant NM were measured, and it was found that a similar pattern of behavior as for the peptide array results, although the magnitude of changes in the two experiments are not linearly related (FIG. 3e). It was also confirmed that the biological relevance of these results by measuring the frequency of prion induction when WT Ca NM fused to YFP is overexpressed in S. cerevesiae in which the endogenous prion domain of Sup35 was replaced with WT or mutant Ca NM. Western blot analysis revealed that the expression levels of WT and mutant forms of Ca Sup35 were similar to WT Sc Sup35 (FIG. 9). It was found that mutations in the Ca interaction site also reduce the frequency of prion induction as predicted by the peptide array and seeding results (FIG. 3f). These results confirm that the Ca interaction site identified by peptide array analysis is involved in transmission of prion conformers, and that changes in the transmission efficiency can be related to changes in the affinity of Ca NM for a small sequence element.

Role of Local Interactions in the Formation of Prion Strains

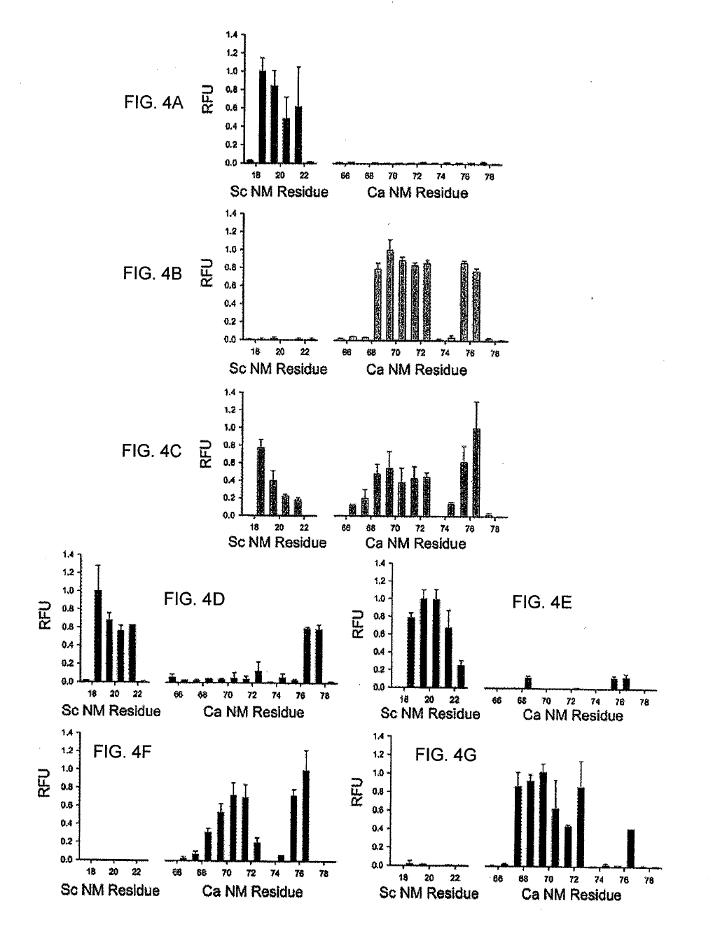

Given the importance of Sup35 interaction sites in governing the conformational conversion of the corresponding prion domains, it was wondered if these regions may also be involved in the formation of distinct aggregate conformations that can possess profoundly different abilities to cross transmission barriers. Specifically, the mechanism was investigated by which a promiscuous Sc/Ca Sup35 chimera (Sc 1-40, Ca 49-143, Sc 124-253) that can cross the Sc/Ca Sup35 transmission barrier is able to assemble into distinct strains that can selectively seed one prion domain versus the other^{17,27,30}. Importantly, both the Sc and Ca interaction sites are located within the sequence of the Sc/Ca chimera. It was found that while each prion domain is only able to associate with its own interaction site (FIGS. 4a and 4b), the chimera is able to associate with both the Sc and Ca interaction sites (FIG. 4c). Since a mixture of chimera strains form during spontaneous assembly or specific strains form by seeding with either prion domain, it was suspected that intermolecular interactions between these interaction sites initiated the formation of a specific prion strain. In support of this hypothesis, chimera mutants prone to spontaneous form fibers that selectively seeded one prion domain versus the other were analyzed. Remarkably, it was found that mutations (G70, 71, 80, 81A) in the Ca portion of the chimera reduce binding to the Ca interaction site but have little effect on binding to the Sc interaction site (FIG. 4d). Conversely, it was found that a mutant (S17R) in the Sc portion of the chimera significantly reduces its binding to the Sc interaction site but has little effect on its binding to the Ca interaction site (FIG. 4e). These results correlate well with in vitro and in vivo results showing that the G70, 71, 80, 81A chimera mutant prefers to assemble into fibers that selectively seed Sc NM, while the S17R chimera mutant prefers to assemble into fibers that selectively seed Ca NM³⁰.

Similar results were obtained using temperature to influence the formation of distinct structural strains of the “wild-type” chimera. While the WT chimera at 25° C. can interact with peptides in both interaction sites (FIG. 4c), it was found that the chimera at 4° C. interacts selectively with peptides in the Sc interaction site relative to Ca interaction site (FIG. 4f). This correlates with the preference of the Sc/Ca chimera to spontaneously assemble into a structural strain at 15° C. that seeds Sc NM selectively both in vitro and in vivo^{27,30}, which was also confirmed in vitro at 4° C. when the chimera fibers were formed at low protein concentrations (equal to or less than 1 μM; data not shown). At 37° C. it is found that the chimera interacts selectively with peptides in the Ca interaction site relative to the Sc interaction site (FIG. 4g). This selectivity correlates well with the preference of the chimera to spontaneously assemble into fibers that selectively seed Ca NM both in vitro and in vivo^{27,30}. Taken collectively, these results suggest that strains initiated by specific sequence elements possess the specificity to selectively seed proteins with the corresponding recognition sequences.

Sequence elements were identified within two Sup35 variants that govern their replication and showed how these sequence elements dictate the nature of species transmission barriers and the formation of distinct prion strains. The first attempt to identify such sequence elements was to identify residues in self-contact that may strongly influence the assembly behavior of the prion domains. Despite the success of this approach of using pyrene excimer fluorescence for identifying important sequence elements in Sc NM⁴⁵, the experiments were less successful for Ca NM, although subsequent peptide array analysis confirmed that Ca NM does contain discrete sequence elements that control its assembly. High resolution techniques such as solid-state NMR are just beginning to yield structural models for the best studied amyloids, such as for Aβ fibers⁴⁷⁻⁴⁹. The technique described herein provides an effective way to determine functional properties of amyloid fibers, such as which residues are of considerable importance for replication and seeding and how do different structural strains selectively seed specific proteins.

It was also found that each prion domain recognized only a small subset of peptides, and only peptides from their own prion domains, in a highly specific manner. Both identified sequences are rich in glutamine, asparagine, tyrosine and glycine residues, which make up more than 85% of the residues in both interaction sites (60 vs. 55% Q and N, 16 vs. 18% Y, 12 vs. 14% G for Sc and Ca Sup35), but this is similar to the overall composition of each N-terminal domain (54 vs. 51% Q and N, 16 vs. 13% Y, 17 vs. 14% G for Sc and Ca Sup35). However, one distinguishing feature of both interaction sites is that they are located adjacent to the repeat sequences within the prion domains. The Sc interaction site that spans residues 8-32 is close to the start of 5.5 imperfect oligopeptide repeats (P/QGGYQQ/YN) (SEQ ID NO: 264) that span residues 43-97. Likewise, the Ca interaction site that spans residues 59-86 overlaps with the beginning of 6 imperfect repeats (RGGYQQ/YNN) (SEQ ID NO: 265) that span residues 70-132. Importantly, the sequences of the oligopeptide repeats are remarkably similar. It is speculated that during seeding or nucleation residues in the interaction sites act as initiation elements by associating with residues either presented at the ends of growing fibers or within another monomer; this interaction may trigger the structuring of the neighboring repeat sequences. This hypothesis is supported by experiments showing that a mutant of Sc NM without the first 40 residues adjacent to the oligopeptide repeats is severely defective in both nucleation and seeding in vitro and in vivo. Interestingly, it has also been observed genetically that sequence elements near similar oligopeptide repeats in another yeast prion, New1, are essential for its aggregation and propagation^{50,51}.

Based on the identification of the Sup35 interaction sites, some of the first molecular details about how different prion strains form is provided. Unlike protein folding where each sequence encodes a unique protein structure, the misfolding of proteins into amyloid fibers often results in formation of several different aggregate conformations. It is found that the initial intermolecular interactions between prions govern their structural strains, and that the energy landscape governing the preferred structural strain can be controlled by small sequence elements. It has been proposed that the molecular basis for one structural strain to predominately form at any given condition is that once a specific structure is nucleated it can propagate with extremely fast kinetics relative to the kinetics of nucleation of different strains. In agreement with this proposed mechanism, it is found that the specificity of the Sup35 chimera for either interaction site, governed both by mutations or environmental changes, was transient and was lost after long incubation times. This is consistent with the Sup35 interaction sites being involved in an initial intermolecular interaction that establishes a specific conformation which is efficiently propagated, preventing less energetically-favorable conformations from being sampled.

Prion strains and transmission barriers have long been realized to be closely related, but a clear molecular explanation for this relationship has been elusive. Results are provided that suggest a simple molecular explanation for this relationship for a promiscuous Sc/Ca Sup35 chimera: intermolecular interactions between discrete sequence elements initiate an aggregate conformation that selectively seeds proteins with an identical recognition sequence. This explanation also provides a connection between prion sequence, the primary determinant of transmission barriers, and aggregate structure. Further, since seeding is governed by intermolecular interactions, it seems reasonable that the seeding specificity of a structural strain could be governed by intermolecular interactions between discrete specificity elements. Interestingly, it is often observed that prion aggregates, particularly those of PrP, from a given species adapt and change their overall conformation when they are introduced into an organism with a different prion sequence^{41,52-54}. It may be that sequence differences between aggregated and soluble proteins at critical interaction sites may disfavor the same strain due to side chain mismatches and favor seeding with different interaction sites that lead to adapted structural strains.

FIG. 11 is a schematic illustration of an exemplary peptide analysis method described herein. A plurality of 20-mer peptides 1100 each include a double lysine tag 1101 attached by PEG linker 1102, and are attached at a C-terminal end 1103 to a cellulose membrane 1104. In Step A, the peptides are cleaved from the membrane and printed on a reactive glass slide 1105 (e.g., an aldehyde functionalized glass slide with 3×300-1000 spots per slide). In one embodiment, peptide density is about 15-150 fmol/mm². In Step B, the slide 1105 is blocked for about 1 hr in 3% BSA, 0.1% T₂O. In Step C, denatured NM is prepared and diluted into PBS buffer. For example, a sample chamber 1106 can contain a solution 1107 of NM 230C ALEXA FLUOR® 532 and CA NM 227C ALEXA FLUOR® 647, 5% labeling ratio. In Step D, the slide 1105 (e.g., array) is placed in the chamber 1106 and the labeled prion domains in the solution 1107 are allowed to hybridize without rotation. In Step E, the array is removed from the chamber 1106 and washed with 2% SDS. The array is subsequently imaged at 532 and 635 nm.

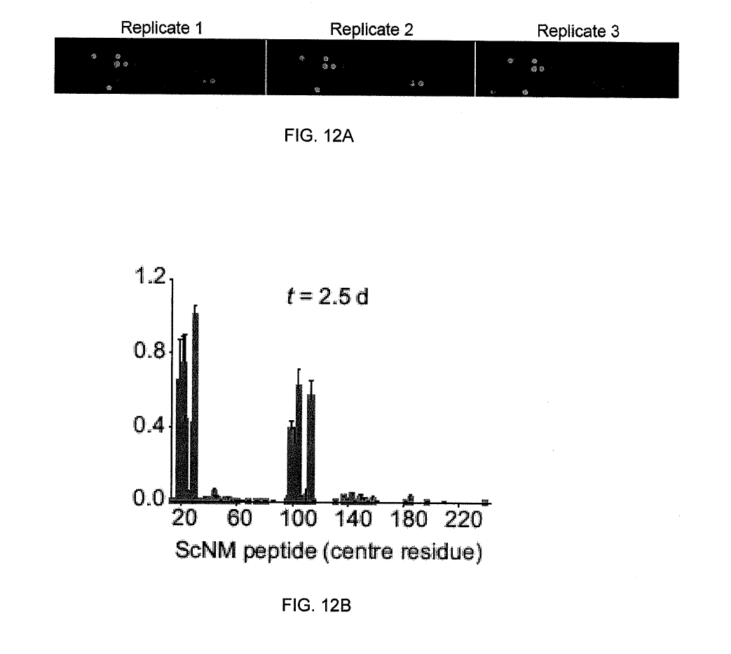

FIGS. 12a-12b are illustrations of the identification of recognition sequences within ScNM using peptide arrays. (See also FIGS. 1a-1g and related text.) To determine if other peptide regions could interact with ScNM, albeit with lower efficiency, the fact that the spontaneous assembly of the full-length protein is very slow in quiescent reactions, even at fivefold higher concentration, was taken advantage of. At this concentration and with a higher fraction of the protein carrying the fluorescent probe (75% versus 5% of protein), label could be detected at a second set of peptides, spanning residues 90-120 after one to two days (FIG. 12b, SEQ ID NO: 3-133). This region corresponded to the second previously identified site of intermolecular contact within mature ScNM fibers (85-105). The reactivity of the surface-bound peptides indicates that these regions are not only sites of intermolecular contact in mature fibers but also represent highly specific self-recognition elements within soluble molten, non-prion conformers.

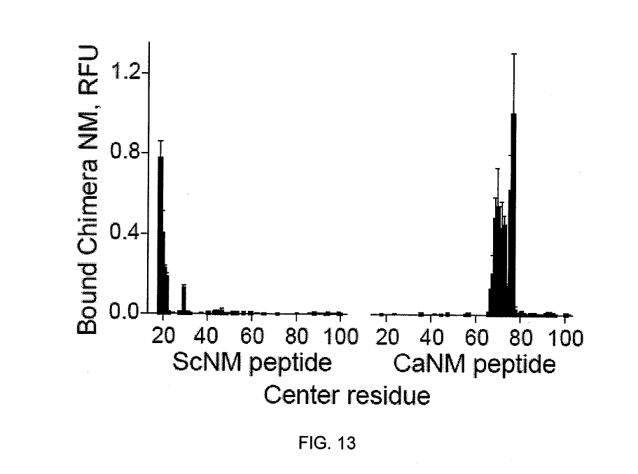

FIG. 13 illustrates an analysis of CaNM recognition sequences and the species barrier between Sc/Ca NM. (See also FIGS. 1a-1g and related text.) A promiscuous Sc/Ca NM chimaera was employed that has been shown previously to traverse the species barrier between S. cerevisiae and C. albicans. This chimeric protein contains segments from both ScNM and CaNM (residues: S. cerevisiae, 1-40 and 124-253; C. albicans, 49-141; (SEQ ID NO: 1-3). Incubating the full-length NM chimaera with an array displaying libraries of both ScNM and CaNM peptides revealed that it was able to interact with the prion recognition elements from both species in a highly specific manner.

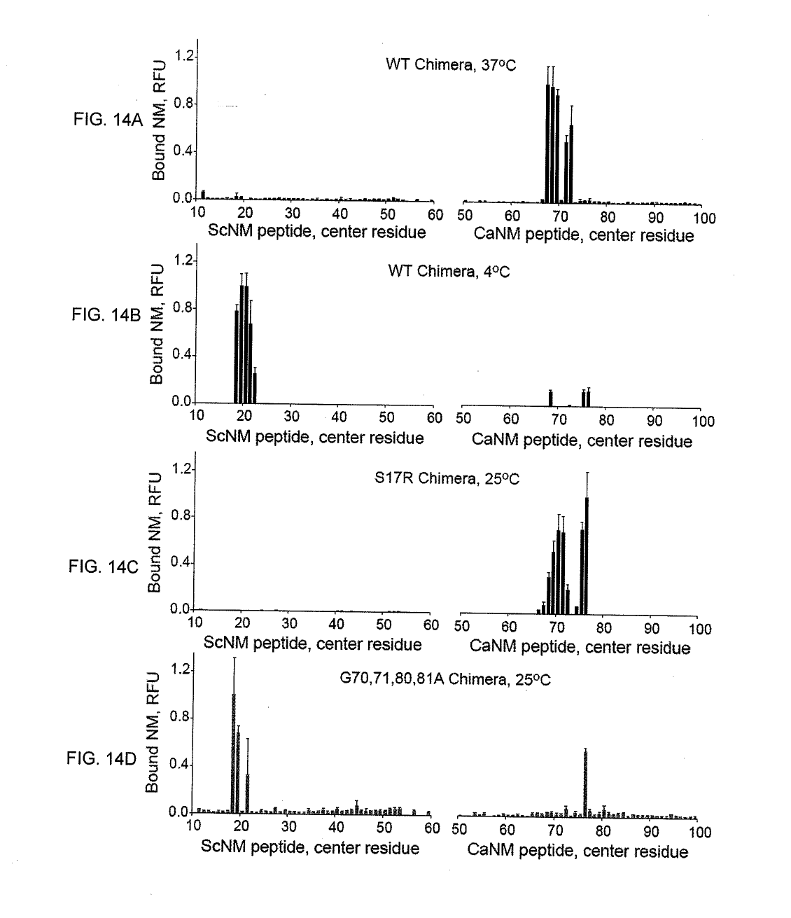

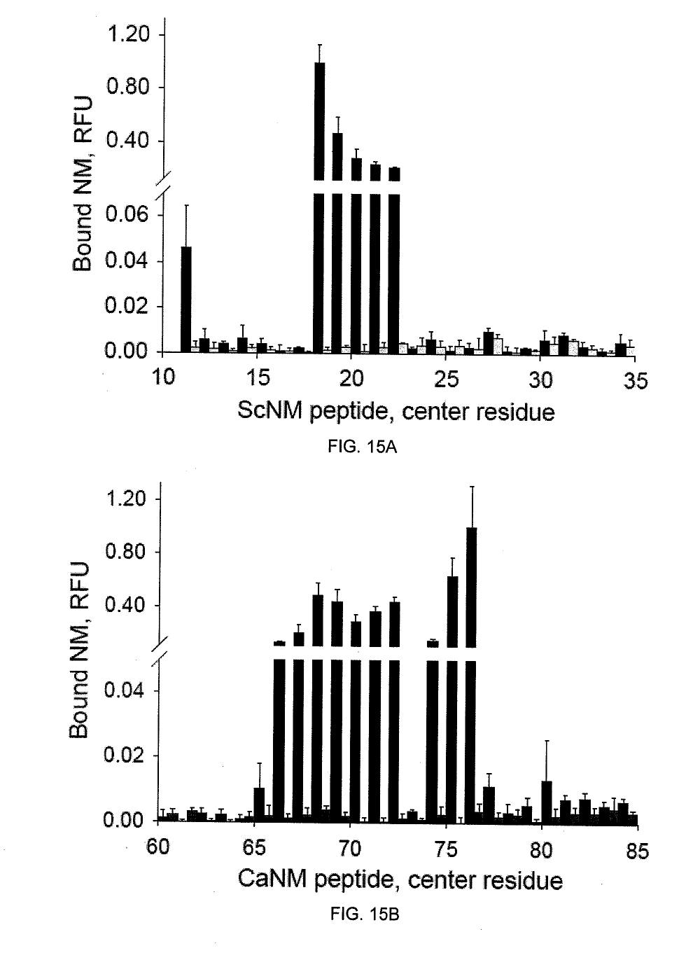

FIGS. 14a-14d illustrate an analysis of the conformational preference of the Sc/Ca NM chimaera. As described above, when the chimeric protein was incubated with the peptide arrays at 25 uC, it interacted with peptides from both species (FIG. 13). However, at 37 uC it interacted selectively with CaNM peptides (FIG. 14a). At 4uC it interacted selectively with ScNM peptides (FIG. 14b). Thus, the ability of the chimeric protein to assemble into distinct species-specific strains at different temperatures is enciphered by the same small sequence elements that nucleate amyloid assembly. Next, the hypothesis that the effects of mutations on the formation of species-specific strains could be explained by these same prion recognition elements was tested. As previously reported, changing serine residue 17 to arginine (S17R) in the Sc/Ca chimaera favors assembly of a prion strain that selectively seeds CaNM36. Conversely, changing four glycines at positions 70, 71, 80 and 81 to alanines (4G/A) favors assembly of a strain that selectively seeds ScNM36. When the S17R chimaera was incubated with the arrays at 25 uC, binding to all ScNM peptides was reduced to background (FIG. 14c). Importantly, binding to CaNM peptides was unaffected (FIG. 14c). Similarly, when the 4G/A chimaera was incubated with the arrays, binding to all but one of the CaNM peptides was greatly reduced but binding to ScNM peptides was similar to the original chimaera (FIG. 14d).

FIGS. 15a-15b illustrate an analysis of the mutational disruption of the ScNM and CaNM recognition elements. Mutations in the Sc/Ca chimaera might alter prion recognition and strain formation by biasing the conformations sampled by the molten full-length proteins such that particular recognition elements are masked. Alternatively, they might directly interfere with interactions between the full-length proteins and their cognate recognition elements. To investigate how the ability of the original chimeric protein to interact with arrays containing mutant peptides was tested. Labelled chimeric protein bound robustly to the wild-type ScNM and CaNM peptides (dark bars, FIGS. 15a and 15b). However, it exhibited no interaction above background with any ScNM peptides containing the 517R mutation (light bars, FIG. 15a) or any CaNM peptides containing the 4G/A mutations (light bars, FIG. 15b). Because the original chimaera reacted with wild-type peptides on the same array, it must have displayed its own recognition element. Its inability to interact with the mutant peptides, therefore, indicates that the mutations directly disrupt the recognition function of the sequence elements rather than solely altering the conformations of the soluble protein. Thus, these mutations, which bias prions toward the formation of distinct strains and alter cross-species prion transmission, do so by directly interfering with recognition of the prion specificity elements.

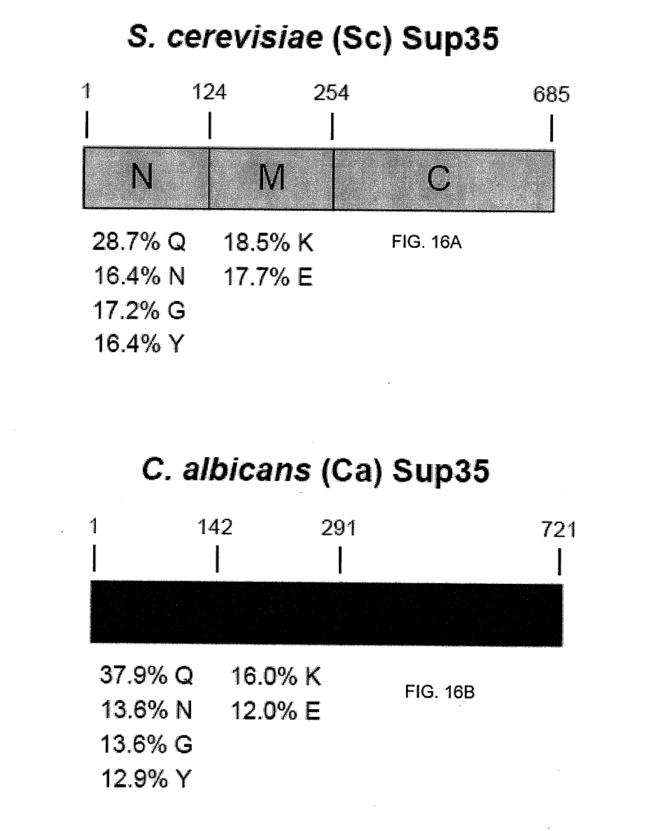

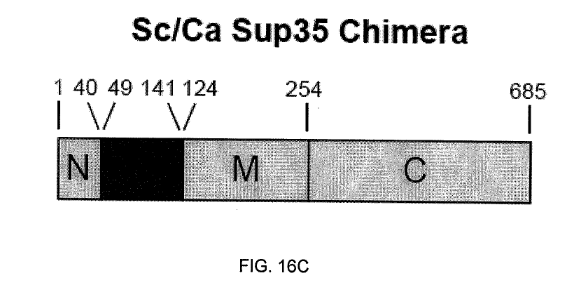

FIGS. 16a-16c are diagrams showing the molecular architectures and amino-acid sequences of three Sup35 constructs. SEQ ID NO: 1 corresponds to FIG. 16a, SEQ ID NO: 2 corresponds to FIG. 16b, and SEQ ID NO: 3 corresponds to FIG. 16c.

SEQ ID NO: 1-3 are amino acid sequences of three Sup35 constructs described herein.

SEQ ID NO: 4-133 are amino acid sequences of ScNM 20mer peptides that were tested in this work. Peptides having the SEQ ID NO: 11-14 and 22 bound full-length ScNM after 2 hours (1 μM ScNM, 5% label) of incubation. Peptides having the SEQ ID NO: 5, 15, 21, 74, 76, 77, 79, and 88 bound full-length ScNM after 2.5 days (5 μM ScNM, 75% label) of incubation.

SEQ ID NO: 134-263 are amino acid sequences of CaNM 20mer peptides that were tested in this work. SEQ ID NO: 163-167, 170, and 171 bound full-length ScNM after 2 hours (1 μM ScNM, 5% label) of incubation. SEQ ID NO: 151, 160-163, 170, 175-177, 213, 214, and 217 bound full-length ScNM after 2.5 days (5 μM ScNM, 75% label) of incubation.

Methods

Mutagenesis, protein purification and cysteine labeling. Single cysteine and arginine mutations were introduced into NM using QUIKCHANGE™ mutagenesis (Stratagene). All Sc and Ca NM constructs contained a C-terminal 7×His tag (SEQ ID NO: 266), as did the Sc/Ca chimera constructs except for S17R which contained a 6×His tag (SEQ ID NO: 267). All NM proteins were purified as described previously⁵⁵ except that the proteins were eluted from the Ni-NTA column using low pH instead of imidazole. The NM cysteine mutants at or near the C-terminus (Sc A230C, Ca S227C and all chimeras at extreme C-terminus) were labeled overnight with maleimide-functionalized ALEXA FLUOR® 555 or 647 (Invitrogen) using a 5:1 to 10:1 molar ratio of label:monomer at room temperature, and the free label was removed using a Ni-NTA column. Ca NM cysteine mutants were labeled overnight with pyrene maleimide at a ratio of label:monomer of 10:1 at room temperature, and the free label was removed in the same way as for the ALEXA FLUOR® dyes.

Peptide array synthesis, hybridization and quantification. The peptides were synthesized on modified cellulose membranes using SPOT™ technology⁵⁶ (JPT Peptide Technologies GmbH). Each peptide contained a double alanine tag at its N-terminus, 20 residues from the prion domains, a hydrophilic linker (1-amino-4,7,10-trioxa-13-tridecanamine succinimic acid⁵⁷) and a double lysine tag at its C-terminus. The peptides were cleaved off the membranes, freeze dried and resuspended in buffer (40% DMSO, 5% glycerol, 55% PBS, pH 9) for printing. The peptides were then printed onto hydrogel glass slides (NEXTERION® Slide H, Schott) functionalized with reactive NHS ester moieties. Each peptide spot (250 μM in diameter) was printed with 3 drops of 0.5 nL of peptide solution at a concentration of approximately 2.5 μM using non-contact printing (JPT Peptide Technologies GmbH). The unreacted peptides were removed from the hydrogel slides, dried and then the slides were blocked with 3% BSA in PBST for 1 hr. The NM proteins were denatured in 6 M GuHCl at 100° C. for approximately 20 minutes, diluted 125 times in PBST containing 3% BSA to a final concentration of 1-5 μM and a label ratio of 5-75%. A single peptide array was incubated with approximately 2-3 mL of diluted NM using an ATLAS™ hybridization chamber (BD Biociences) without mixing for a given period of time. The peptide arrays were then washed 5 times with 50 mL of 2% SDS for 30 minutes, 5 times with 50 mL of water, 3 times with 50 mL of methanol and then spun dry. The methanol washes were found to not be essential but helped prevent uneven drying of the slides. The arrays were then imaged using a GENEPIX® 4000A scanner and the median values for the peptide spots of two to three replicates were quantified using GENEPIX® Pro 6.0 software (Molecular Devices).

In vitro nucleation and seeding assays. Unseeded and seeded reactions were performed using either a Molecular Devices SPECTRAMAX® M2 or a Tecan SAFIRE²™ plate reader. For unseeded reactions black microtiter plates (MICROFLUORI®, Thermo Labsystems) containing a single 4 mm glass bead per well were blocked with 2.5 mg/mL of ovalbumin in PBS with 40 μM Thioflavin T (ThT) for several hours. After the blocking solution was removed, denatured NM was diluted to 4 μM in PBS containing 40 μM ThT and loaded into the microtiter plate. Each plate was mixed for 10 sec/min and the assembly kinetics were monitored by ThT fluorescence at 482 nm (excited at 450 nm). Similar experiments were performed using maleimide-activated microtiter plates (Pierce) that were coated with Sc NM 20mer peptides. Briefly, 20mer peptides containing an N-terminal cysteine and short PEG spacer (MW 0.39 kD) were dissolved in DMSO and incubated in maleimide-functionalized microtiter plates overnight at 100 μM (10% DMSO, 5.4 M GuHCl, 90 mM potassium phosphate, pH 7.2). The wells were washed extensively with reaction buffer, blocked with 3% BSA for several hours and unseeded reactions were conducted as described above. Seeded reactions were performed with unblocked microtiter plates without mixing beads, and the plates were typically mixed for 3 sec/min. The seeding kinetics were monitored by both ThT fluorescence (once per minute) and SDS resistance (endpoint) for approximately 45 minutes.

In vivo prion induction experiments. Integrating plasmids containing WT Ca NM and various point mutants fused to the C-terminus of Sc Sup35 were used to replace the endogenous Sup35 allele as described elsewhere⁴⁵. The S. cerevesiae strain used in this work was 74D-694 [MATa, his3, leu2, trp1, ura3; suppressible marker ade1-14(UGA)]. YFP and WT Ca NM fused to YFP were overexpressed from a 2μ plasmid under the control of the GAL1 promoter. After 16 hrs of growth in 2% galactose the cells were plated onto SD and SD-Ade plates and the number of colonies were quantified after 3 (SD) or 7 (SD-Ade) days to determine the fraction of cells converted to the prion state.

Other Information

Pyrene excimer fluorescence. The fraction of Ca NM labeled with pyrene, determined by absorbance at 278 nm and BCA analysis (Pierce), was adjusted to 50% with unlabeled NM and converted into fibers at 2.5 μM NM using 5% WT Ca NM fibers. The excimer ratio was measured as the ratio of fluorescence at 465 nm relative to the fluorescence at 390 nm using a SpectraMax2 platereader (Molecular Probes) for the labeled fibers relative to labeled monomer. The exact position of the reference peak was found to be dependent on the fluorimeter and bandwidth used, so care should be taken when normalizing excimer fluorescence.

Additional details of the experiments are provided in the accompanying manuscript which is a part of this specification.

### REFERENCES

1. Litvinovich, S. V. et al. Formation of amyloid-like fibrils by self-association of a partially unfolded fibronectin type III module. Journal of Molecular Biology 280, 245-258 (1998).
2. Guijarro, J. I., Sunde, M., Jones, J. A., Campbell, I. D. & Dobson, C. M. Amyloid fibril formation by an SH3 domain. Proceedings of the National Academy of Sciences of the United States of America 95, 4224-4228 (1998).
3. Fandrich, M., Fletcher, M. A. & Dobson, C. M. Amyloid fibrils from muscle myoglobin—Even an ordinary globular protein can assume a rogue guise if conditions are right. Nature 410, 165-166 (2001).
4. Si, K. et al. A neuronal isoform of CPEB regulates local protein synthesis and stabilizes synapse-specific long-term facilitation in Aplysia. Cell 115, 893-904 (2003).
5. Si, K., Lindquist, S. & Kandel, E. R. A neuronal isoform of the Aplysia CPEB has prion-like properties. Cell 115, 879-891 (2003).
6. Chapman, M. R. et al. Role of Escherichia coli curli operons in directing amyloid fiber formation. 295, 851-855 (2002).
7. Fowler, D. M. et al. Functional amyloid formation within mammalian tissue. Plos Biology 4, 100-107 (2006).
8. True, H. L. & Lindquist, S. L. A yeast prion provides a mechanism for genetic variation and phenotypic diversity. 407, 477-483 (2000).
9. True, H. L., Berlin, I. & Lindquist, S. L. Epigenetic regulation of translation reveals hidden genetic variation to produce complex traits. 431, 184-187 (2004).
10. Prusiner, S. B. Prions. Proceedings of the National Academy of Sciences of the United States of America 95, 13363-13383 (1998).
11. Shorter, J. & Lindquist, S. Prions as adaptive conduits of memory and inheritance. Nature Reviews Genetics 6, 435-450 (2005).
12. Tuite, M. F. & Cox, B. S. Propagation of yeast prions. Nature Reviews Molecular Cell Biology 4, 878-889 (2003).
13. Uptain, S. M. & Lindquist, S. Prions as protein-based genetic elements. Annual Review of Microbiology 56, 703-741 (2002).
14. Chien, P., Weissman, J. S. & DePace, A. H. Emerging principles of conformation based prion inheritance. Annual Review of Biochemistry 73, 617-656 (2004).
15. Eaglestone, S. S., Cox, B. S. & Tuite, M. F. Translation termination efficiency can be regulated in Saccharomyces cerevisiae by environmental stress through a prion-mediated mechanism. Embo Journal 18, 1974-1981 (1999).
16. Chemoff, Y. O. et al. Evolutionary conservation of prion-forming abilities of the yeast Sup35 protein. Molecular Microbiology 35, 865-876 (2000).
17. Chien, P. & Weissman, J. S. Conformational diversity in a yeast prion dictates its seeding specificity. Nature 410, 223-227 (2001).
18. Santoso, A., Chien, P., Osherovich, L. Z. & Weissman, J. S. Molecular basis of a yeast prion species barrier. Cell 100, 277-288 (2000).
19. Resende, C. et al. The Candida albicans Sup35p protein (CaSup35p): function, prion-like behaviour and an associated polyglutamine length polymorphism. Microbiology-Sgm 148, 1049-1060 (2002).
20. Nakayashiki, T., Ebihara, K., Bannai, H. & Nakamura, Y. Yeast PSI+ “prions” that are crosstransmissible and susceptible beyond a species barrier through a quasi-prion state. Molecular Cell 7, 1121-1130 (2001).
21. Kushnirov, V. V., Kochneva-Pervukhova, N., Chechenova, M. B., Frolova, N. S. & Ter-Avanesyan, M. D. Prion properties of the Sup35 protein of yeast Pichia methanolica. Embo Journal 19, 324-331 (2000).
22. Scott, M. et al. Transgenic Mice Expressing Hamster Prion Protein Produce Species-Specific Scrapie Infectivity and Amyloid Plaques. Cell 59, 847-857 (1989).
23. Prusiner, S. B. et al. Transgenetic Studies Implicate Interactions between Homologous Prp Isoforms in Scrapie Prion Replication. Cell 63, 673-686 (1990).
24. Hill, A. F. et al. Species-barrier-independent prion replication in apparently resistant species. Proceedings of the National Academy of Sciences of the United States of America 97, 10248-10253 (2000).
25. Scott, M. et al. Propagation of Prions with Artificial Properties in Transgenic Mice Expressing Chimeric Prp Genes. Cell 73, 979-988 (1993).
26. Tanaka, M., Chien, P., Naber, N., Cooke, R. & Weissman, J. S. Conformational variations in an infectious protein determine prion strain differences. Nature 428, 323-328 (2004).
27. Tanaka, M., Chien, P., Yonekura, K. & Weissman, J. S. Mechanism of cross-species prion transmission: An infectious conformation compatible with two highly divergent yeast prion proteins. Cell 121, 49-62 (2005).
28. Palmer, M. S., Dryden, A. J., Hughes, J. T. & Collinge, J. Homozygous Prion Protein Genotype Predisposes to Sporadic Creutzfeldt-Jakob Disease. Nature 352, 340-342 (1991).
29. Collinge, J., Palmer, M. S. & Dryden, A. J. Genetic Predisposition to Iatrogenic Creutzfeldt-Jakob Disease. Lancet 337, 1441-1442 (1991).
30. Chien, P., DePace, A. H., Collins, S. R. & Weissman, J. S. Generation of prion transmission barriers by mutational control of amyloid conformations. Nature 424, 948-951 (2003).
31. Supattapone, S. et al. Prion protein of 106 residues creates an artificial transmission barrier for prion replication in transgenic mice. Cell 96, 869-878 (1999).
32. Scott, M. R. et al. Identification of a prion protein epitope modulating transmission of bovine spongiform encephalopathy prions to transgenic mice. Proceedings of the National Academy of Sciences of the United States of America 94, 14279-14284 (1997).
33. Collinge, J. et al. Unaltered Susceptibility to Bse in Transgenic Mice Expressing Human Prion Protein. Nature 378, 779-783 (1995).
34. Hill, A. F. et al. The same prion strain causes vCJD and BSE. Nature 389, 448-450 (1997).
35. Collinge, J. Prion diseases of humans and animals: Their causes and molecular basis. Annual Review of Neuroscience 24, 519-550 (2001).
36. Bruce, M. E., McConnell, I., Fraser, H. & Dickinson, A. G. The Disease Characteristics of Different Strains of Scrapie in Sinc Congenic Mouse Lines—Implications for the Nature of the Agent and Host Control of Pathogenesis. Journal of General Virology 72, 595-603 (1991).
37. Bessen, R. A. & Marsh, R. F. Biochemical and Physical-Properties of the Prion Protein from 2 Strains of the Transmissible Mink Encephalopathy Agent. Journal of Virology 66, 2096-2101 (1992).
38. Telling, G. C. et al. Evidence for the conformation of the pathologic isoform of the prion protein enciphering and propagating prion diversity. Science 274, 2079-2082 (1996).
39. Bessen, R. A. et al. Nongenetic Propagation of Strain-Specific Properties of Scrapie Prion Protein. Nature 375, 698-700 (1995).
40. Bruce, M. et al. Transmission of Bovine Spongiform Encephalopathy and Scrapie to Mice—Strain Variation and the Species Barrier. Philosophical Transactions of the Royal Society of London Series B-Biological Sciences 343, 405-411 (1994).
41. Peretz, D. et al. A change in the conformation of prions accompanies the emergence of a new prion strain. Neuron 34, 921-932 (2002).
42. Will, R. G., Ironside, J. W., Hornlimann, B. & Zeidler, M. Creutzfeldt-Jakob disease. Lancet 347, 65-66 (1996).
43. King, C. Y. & Diaz-Avalos, R. Protein-only transmission of three yeast prion strains. 428, 319-323 (2004).
44. Bradley, M. E., Edskes, H. K., Hong, J. Y., Widmer, R. B. & Liebman, S. W. Interactions among prions and prion “strains” in yeast. Proceedings of the National Academy of Sciences of the United States of America 99, 16392-16399 (2002).
45. Krishnan, R. & Lindquist, S. L. Structural insights into a yeast prion illuminate nucleation and strain diversity. Nature 435, 765-772 (2005).
46. DePace, A. H., Santoso, A., Hillner, P. & Weissman, J. S. A critical role for amino-terminal glutamine/asparagine repeats in the formation and propagation of a yeast prion. Cell 93, 1241-1252 (1998).
47. Luhrs, T. et al. 3D structure of Alzheimer's amyloid-beta(1-42) fibrils. Proceedings of the National Academy of Sciences of the United States of America 102, 17342-17347 (2005).
48. Petkova, A. T. et al. Self-propagating, molecular-level polymorphism in Alzheimer's beta-amyloid fibrils. Science 307, 262-265 (2005).
49. Petkova, A. T. et al. A structural model for Alzheimer's beta-amyloid fibrils based on experimental constraints from solid state NMR. Proceedings of the National Academy of Sciences of the United States of America 99, 16742-16747 (2002).
50. Osherovich, L. Z., Cox, B. S., Tuite, M. F. & Weissman, J. S. Dissection and design of yeast prions. Plos Biology 2, 442-451 (2004).
51. Osherovich, L. Z. & Weissman, J. S. Multiple GIn/Asn-rich prion domains confer susceptibility to induction of the yeast PSI+ prion. Cell 106, 183-194 (2001).
52. Kimberlin, R. H., Cole, S. & Walker, C. A. Temporary and Permanent Modifications to a Single Strain of Mouse Scrapie on Transmission to Rats and Hamsters. Journal of General Virology 68, 1875-1881 (1987).
53. Bartz, J. C., Bessen, R. A., McKenzie, D., Marsh, R. F. & Aiken, J. M. Adaptation and selection of prion protein strain conformations following interspecies transmission of transmissible mink encephalopathy. Journal of Virology 74, 5542-5547 (2000).
54. Asante, E. A. et al. BSE priors propagate as either variant CJD-like or sporadic CJD-like prion strains in transgenic mice expressing human prion protein. Embo Journal 21, 6358-6366 (2002).
55. Glover, J. R. et al. Self-seeded fibers formed by Sup35, the protein determinant of [PSI+], a heritable prion-like factor of S. cerevisiae. Cell 89, 811-819 (1997).
56. Frank, R. Spot-Synthesis—an Easy Technique for the Positionally Addressable, Parallel Chemical Synthesis on a Membrane Support. Tetrahedron 48, 9217-9232 (1992).
57. Zhao, Z. G., Im, J. S., Lam, K. S. & Lake, D. F. Site-specific modification of a single-chain antibody using a novel glyoxylyl-based labeling reagent. 10, 424-430 (1999).

While the invention has been particularly shown and described with reference to specific embodiments, it should be understood by those skilled in the art that various changes in form and detail may be made therein without departing from the spirit and scope of the invention as defined by the appended claims.

What is claimed is:

1. A peptide comprising at least 15 contiguous amino acids located between amino acids 1-40 of Sc Sup35, wherein said contiguous amino acids include amino acids 18-22 of Sc Sup35 and assemble with full length Sc Sup35 to form a higher ordered aggregate, and wherein the sequence of said polypeptide does not contain more than 50 contiguous amino acids of the sequence of Sc Sup35 outside the region between amino acids 1-40 of Sc Sup35.

2. The peptide of claim 1, wherein the peptide does not contain more than 20 contiguous amino acids of the sequence of Sc Sup35 outside the region between amino acids 1-40 of Sc Sup35.

3. The peptide of claim 1, wherein the peptide is located between amino acids 8-40 of Sc Sup35.

4. The peptide of claim 1, wherein the peptide is located between amino acids 8-32 of Sc Sup35.

5. The polypeptide of claim 1, wherein the amino acid sequence comprises or consists of amino acids 10-29 of Sc Sup35.

6. The polypeptide of claim 1, wherein the amino acid sequence comprises or consists of amino acids 11-30 of Sc Sup35.

7. The peptide of claim 1, wherein the polypeptide is between 10 and 50 amino acids in length.

8. The peptide of claim 1, wherein the peptide is between 15 and 30 amino acids in length.

9. A peptide at least 90% identical to the peptide of claim 1.

10. A peptide whose sequence differs by not more than 2 amino acid insertions, deletions, or substitutions from that of the peptide of claim 1.

11. A polypeptide whose amino acid sequence comprises a first portion that comprises the peptide of any one of claims 1-10.

12. The peptide of claim 1, further comprising a second portion, wherein the second portion has a biological or chemical activity of interest or comprises a detectable, selectable, or reactive moiety.

13. A higher order aggregate comprising a peptide of claim 1.

14. The higher order aggregate of claim 13, wherein said higher order aggregate is a fibril.

15. A solid support having the peptide of claim 1 covalently or noncovalently attached thereto.

16. A peptide comprising at least 15 contiguous amino acids located between amino acids 59-86 of Ca Sup35, wherein the contiguous amino acids include amino acids 69-76 of Ca Sup35 and assemble with full length Ca Sup35 to form a higher ordered aggregate, and wherein the sequence of said polypeptide does not contain more than 50 contiguous amino acids of the sequence of Ca Sup35 outside the region between amino acids 59-86 of Sc Sup35.

17. The peptide of claim 1, wherein the peptide does not contain more than 20 contiguous amino acids of the sequence of Ca Sup35 outside the region between amino acids 59-86 of Sc Sup35.

18. The peptide of claim 16 wherein the polypeptide is between 10 and 50 amino acids in length.

19. The peptide of claim 16, wherein the peptide is between 15 and 30 amino acids in length.

20. A peptide at least 90% identical to the peptide of claim 16.

21. A peptide whose sequence differs by not more than 2 amino acid insertions, deletions, or substitutions from that of the peptide of claim 16.

22. A polypeptide whose amino acid sequence comprises a first portion that comprises the peptide of any one of claims 16-21.

23. The polypeptide of claim 16, further comprising a second portion, wherein the second portion has a biological or chemical activity of interest or comprises a detectable, selectable, or reactive moiety.

24. A higher order aggregate comprising a peptide of claim 16.

25. The higher order aggregate of claim 24, wherein said higher order aggregate is a fibril.

26. A solid support having the peptide of claim 16 covalently or noncovalently attached thereto.

27. A collection comprising at least 10 different peptides, wherein the peptides are fragments of a polypeptide, wherein the polypeptide is a polypeptide that misfolds or spontaneously aggregates into a higher order structure under appropriate conditions.

28. The collection of claim 27, wherein the peptides scan across between 20% and 100% of the polypeptide and wherein the N-terminal amino acids of the peptides are located between 1 and 10 amino acids from each other within the polypeptide sequence.

29. The collection of claim 27, wherein the peptides are derived from a polypeptide selected from the group consisting of: Sup35 proteins, Ure2 proteins, New1 proteins, Rnq1 proteins, mammalian prion proteins, amyloid precursor protein, Aβ40, Aβ42, immunoglobulin (Ig) light chain, serum amyloid A, wild type or variant transthyretin, lysozyme, BnL, cystatin C, β2-microglobulin, apoliprotein A1, gelsolin mutants, lactotransferrin, islet amyloid polypeptide, fibrinogen, prolactin, insulin, calcitonin, atrial natriuretic factor, α-synuclein, Huntingtin, superoxide dismutase, and α1-chymotrypsin.

30. An array comprising a plurality of peptides, wherein the peptides are fragments of a polypeptide, wherein the polypeptide is a polypeptide that misfolds or spontaneously aggregates into a higher order structure under appropriate conditions.

31. The array of claim 30, wherein the peptides are derived from a polypeptide selected from the group consisting of: Sup35 proteins, Ure2 proteins, New1 proteins, Rnq1 proteins, mammalian prion proteins, amyloid precursor protein, Aβ40, Aβ42, immunoglobulin (Ig) light chain, serum amyloid A, wild type or variant transthyretin, lysozyme, BnL, cystatin C, β2-microglobulin, apoliprotein A1, gelsolin mutants, lactotransferrin, islet amyloid polypeptide, fibrinogen, prolactin, insulin, calcitonin, atrial natriuretic factor, α-synuclein, Huntingtin, superoxide dismutase, and α1-chymotrypsin.

32. A method of forming a higher ordered aggregate comprising the steps of:
providing a composition comprising (a) a peptide comprising a protein aggregation domain and (b) a polypeptide comprising the protein aggregation domain; and
maintaining the composition for a time sufficient for formation of a higher ordered aggregate.

33. The method of claim 32, wherein the peptide is between 15 and 50 amino acids in length.

34. The method of claim 32, wherein the polypeptide is selected from the group consisting of: Sup35 proteins, Ure2 proteins, New1 proteins, Rnq1 proteins, mammalian prion proteins, amyloid precursor protein, Aβ40, Aβ42, immunoglobulin (Ig) light chain, serum amyloid A, wild type or variant transthyretin, lysozyme, BnL, cystatin C, β2-microglobulin, apoliprotein A1, gelsolin mutants, lactotransferrin, islet amyloid polypeptide, fibrinogen, prolactin, insulin, calcitonin, atrial natriuretic factor, α-synuclein, Huntingtin, superoxide dismutase, and α1-chymotrypsin.

35. The method of claim 32, wherein the peptide is attached to a solid support.

36. The method of claim 35, further comprising removing the higher ordered aggregate from the solid support.

37. A method of identifying an aggregation domain of a polypeptide comprising the steps of:
providing an array comprising a plurality of peptides, wherein the peptides are fragments of a polypeptide that spontaneously aggregates into a higher order structure under appropriate conditions;
contacting the array with the polypeptide; and
identifying a peptide to which the polypeptide binds, thereby identifying an aggregation domain of the polypeptide.

38. The method of claim 37, wherein the polypeptide is selected from the group consisting of Sup35 proteins, Ure2 proteins, New1 proteins, Rnq1 proteins, mammalian prion proteins, amyloid precursor protein, Aβ40, Aβ42, immunoglobulin (Ig) light chain, serum amyloid A, wild type or variant transthyretin, lysozyme, BnL, cystatin C, β2-microglobulin, apoliprotein A1, gelsolin mutants, lactotransferrin, islet amyloid polypeptide, fibrinogen, prolactin, insulin, calcitonin, atrial natriuretic factor, α-synuclein, Huntingtin, superoxide dismutase, and α1-chymotrypsin.

39. The method of claim 37, wherein the polypeptide is one whose misfolding or aggregation is implicated in mammalian disease.

40. A method of identifying a candidate agent for modulating protein aggregation comprising:
(i) providing a composition comprising an aggregation-prone polypeptide, a test agent, and a peptide derived from the aggregation-prone polypeptide, wherein the peptide is capable of binding to the aggregation-prone polypeptide in the absence of the test agent; and
(ii) identifying the agent as a candidate agent for modulating protein aggregation if presence of the test agent alters the extent or rate of binding of the peptide and the polypeptide.

41. A method for identifying a candidate agent for inhibiting protein aggregation comprising:
(i) providing a composition comprising an aggregation-prone polypeptide, a test agent, and a peptide derived from the aggregation-prone polypeptide, wherein the peptide is capable of binding to the aggregation-prone polypeptide in the absence of the test agent; and
(ii) identifying the agent as a candidate agent for inhibiting protein aggregation if presence of the test agent reduces the binding of the peptide and the polypeptide.


SEQUENCE LISTING (267 sequences)

SEQ ID NO: 1 - protein, 123 aa, Saccharomyces cerevisiae
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQG

SEQ ID NO: 2 - protein, 141 aa, Candida albicans
MSDQQNTQDQLSGAMANASLNGDQSKQQQQQQQQQQQNYYNPNAAQSFVPQGGYQQFQQFQPQQQQQQYGGYNQYNQYQGGYQQNYNNRGGYQQGYNNRGGYQQNYNNRGGYQGYNQNQQYGGYQQYNSQPQQQQQQQSQG

SEQ ID NO: 3 - protein, 135 aa, Artificial Sequence
  Description of Artificial Sequence Synthetic protein construct
MSDSNQGNNQQNYQQYSQNGNQQQGNNRYQGYQAYNAQAQSFVPQGGYQQFQQFQPQQQQQQYGGYNQYNQYQGGYQQNYNNRGGYQQGYNNRGGYQQNYNNRGGYQGYNQNQQYGGYQQYNSQPQQQQQQQSQG

SEQ ID NO: 4 - protein, 20 aa, Saccharomyces cerevisiae
SDSNQGNNQQNYQQYSQNGN

SEQ ID NO: 5 - protein, 20 aa, Saccharomyces cerevisiae
DSNQGNNQQNYQQYSQNGNQ

SEQ ID NO: 6 - protein, 20 aa, Saccharomyces cerevisiae
SNQGNNQQNYQQYSQNGNQQ

SEQ ID NO: 7 - protein, 20 aa, Saccharomyces cerevisiae
NQGNNQQNYQQYSQNGNQQQ

SEQ ID NO: 8 - protein, 20 aa, Saccharomyces cerevisiae
QGNNQQNYQQYSQNGNQQQG

SEQ ID NO: 9 - protein, 20 aa, Saccharomyces cerevisiae
GNNQQNYQQYSQNGNQQQGN

SEQ ID NO: 10 - protein, 20 aa, Saccharomyces cerevisiae
NNQQNYQQYSQNGNQQQGNN

SEQ ID NO: 11 - protein, 20 aa, Saccharomyces cerevisiae
NQQNYQQYSQNGNQQQGNNR

SEQ ID NO: 12 - protein, 20 aa, Saccharomyces cerevisiae
QQNYQQYSQNGNQQQGNNRY

SEQ ID NO: 13 - protein, 20 aa, Saccharomyces cerevisiae
QNYQQYSQNGNQQQGNNRYQ

SEQ ID NO: 14 - protein, 20 aa, Saccharomyces cerevisiae
NYQQYSQNGNQQQGNNRYQG

SEQ ID NO: 15 - protein, 20 aa, Saccharomyces cerevisiae
YQQYSQNGNQQQGNNRYQGY

SEQ ID NO: 16 - protein, 20 aa, Saccharomyces cerevisiae
QQYSQNGNQQQGNNRYQGYQ

SEQ ID NO: 17 - protein, 20 aa, Saccharomyces cerevisiae
QYSQNGNQQQGNNRYQGYQA

SEQ ID NO: 18 - protein, 20 aa, Saccharomyces cerevisiae
YSQNGNQQQGNNRYQGYQAY

SEQ ID NO: 19 - protein, 20 aa, Saccharomyces cerevisiae
SQNGNQQQGNNRYQGYQAYN

SEQ ID NO: 20 - protein, 20 aa, Saccharomyces cerevisiae
QNGNQQQGNNRYQGYQAYNA

SEQ ID NO: 21 - protein, 20 aa, Saccharomyces cerevisiae
NGNQQQGNNRYQGYQAYNAQ

SEQ ID NO: 22 - protein, 20 aa, Saccharomyces cerevisiae
GNQQQGNNRYQGYQAYNAQA

SEQ ID NO: 23 - protein, 20 aa, Saccharomyces cerevisiae
NQQQGNNRYQGYQAYNAQAQ

SEQ ID NO: 24 - protein, 20 aa, Saccharomyces cerevisiae
QQQGNNRYQGYQAYNAQAQP

SEQ ID NO: 25 - protein, 20 aa, Saccharomyces cerevisiae
QQGNNRYQGYQAYNAQAQPA

SEQ ID NO: 26 - protein, 20 aa, Saccharomyces cerevisiae
QGNNRYQGYQAYNAQAQPAG

SEQ ID NO: 27 - protein, 20 aa, Saccharomyces cerevisiae
GNNRYQGYQAYNAQAQPAGG

SEQ ID NO: 28 - protein, 20 aa, Saccharomyces cerevisiae
NNRYQGYQAYNAQAQPAGGY

SEQ ID NO: 29 - protein, 20 aa, Saccharomyces cerevisiae
NRYQGYQAYNAQAQPAGGYY

SEQ ID NO: 30 - protein, 20 aa, Saccharomyces cerevisiae
RYQGYQAYNAQAQPAGGYYQ

SEQ ID NO: 31 - protein, 20 aa, Saccharomyces cerevisiae
YQGYQAYNAQAQPAGGYYQN

SEQ ID NO: 32 - protein, 20 aa, Saccharomyces cerevisiae
QGYQAYNAQAQPAGGYYQNY

SEQ ID NO: 33 - protein, 20 aa, Saccharomyces cerevisiae
GYQAYNAQAQPAGGYYQNYQ

SEQ ID NO: 34 - protein, 20 aa, Saccharomyces cerevisiae
YQAYNAQAQPAGGYYQNYQG

SEQ ID NO: 35 - protein, 20 aa, Saccharomyces cerevisiae
QAYNAQAQPAGGYYQNYQGY

SEQ ID NO: 36 - protein, 20 aa, Saccharomyces cerevisiae
AYNAQAQPAGGYYQNYQGYS

SEQ ID NO: 37 - protein, 20 aa, Saccharomyces cerevisiae
YNAQAQPAGGYYQNYQGYSG

SEQ ID NO: 38 - protein, 20 aa, Saccharomyces cerevisiae
NAQAQPAGGYYQNYQGYSGY

SEQ ID NO: 39 - protein, 20 aa, Saccharomyces cerevisiae
AQAQPAGGYYQNYQGYSGYQ

SEQ ID NO: 40 - protein, 20 aa, Saccharomyces cerevisiae
QAQPAGGYYQNYQGYSGYQQ

SEQ ID NO: 41 - protein, 20 aa, Saccharomyces cerevisiae
AQPAGGYYQNYQGYSGYQQG

SEQ ID NO: 42 - protein, 20 aa, Saccharomyces cerevisiae
QPAGGYYQNYQGYSGYQQGG

SEQ ID NO: 43 - protein, 20 aa, Saccharomyces cerevisiae
PAGGYYQNYQGYSGYQQGGY

SEQ ID NO: 44 - protein, 20 aa, Saccharomyces cerevisiae
AGGYYQNYQGYSGYQQGGYQ

SEQ ID NO: 45 - protein, 20 aa, Saccharomyces cerevisiae
GGYYQNYQGYSGYQQGGYQQ

SEQ ID NO: 46 - protein, 20 aa, Saccharomyces cerevisiae
GYYQNYQGYSGYQQGGYQQY

SEQ ID NO: 47 - protein, 20 aa, Saccharomyces cerevisiae
QNYQGYSGYQQGGYQQYNPD

SEQ ID NO: 48 - protein, 20 aa, Saccharomyces cerevisiae
QGYSGYQQGGYQQYNPDAGY

SEQ ID NO: 49 - protein, 20 aa, Saccharomyces cerevisiae
SGYQQGGYQQYNPDAGYQQQ

SEQ ID NO: 50 - protein, 20 aa, Saccharomyces cerevisiae
QQGGYQQYNPDAGYQQQYNP

SEQ ID NO: 51 - protein, 20 aa, Saccharomyces cerevisiae
GYQQYNPDAGYQQQYNPQGG

SEQ ID NO: 52 - protein, 20 aa, Saccharomyces cerevisiae
QYNPDAGYQQQYNPQGGYQQ

SEQ ID NO: 53 - protein, 20 aa, Saccharomyces cerevisiae
PDAGYQQQYNPQGGYQQYNP

SEQ ID NO: 54 - protein, 20 aa, Saccharomyces cerevisiae
GYQQQYNPQGGYQQYNPQGG

SEQ ID NO: 55 - protein, 20 aa, Saccharomyces cerevisiae
QQYNPQGGYQQYNPQGGYQQ

SEQ ID NO: 56 - protein, 20 aa, Saccharomyces cerevisiae
QYNPQGGYQQYNPQGGYQQQ

SEQ ID NO: 57 - protein, 20 aa, Saccharomyces cerevisiae
YNPQGGYQQYNPQGGYQQQF

SEQ ID NO: 58 - protein, 20 aa, Saccharomyces cerevisiae
NPQGGYQQYNPQGGYQQQFN

SEQ ID NO: 59 - protein, 20 aa, Saccharomyces cerevisiae
PQGGYQQYNPQGGYQQQFNP

SEQ ID NO: 60 - protein, 20 aa, Saccharomyces cerevisiae
QGGYQQYNPQGGYQQQFNPQ

SEQ ID NO: 61 - protein, 20 aa, Saccharomyces cerevisiae
GGYQQYNPQGGYQQQFNPQG

SEQ ID NO: 62 - protein, 20 aa, Saccharomyces cerevisiae
GYQQYNPQGGYQQQFNPQGG

SEQ ID NO: 63 - protein, 20 aa, Saccharomyces cerevisiae
YQQYNPQGGYQQQFNPQGGR

SEQ ID NO: 64 - protein, 20 aa, Saccharomyces cerevisiae
QQYNPQGGYQQQFNPQGGRG

SEQ ID NO: 65 - protein, 20 aa, Saccharomyces cerevisiae
QYNPQGGYQQQFNPQGGRGN

SEQ ID NO: 66 - protein, 20 aa, Saccharomyces cerevisiae
YNPQGGYQQQFNPQGGRGNY

SEQ ID NO: 67 - protein, 20 aa, Saccharomyces cerevisiae
NPQGGYQQQFNPQGGRGNYK

SEQ ID NO: 68 - protein, 20 aa, Saccharomyces cerevisiae
PQGGYQQQFNPQGGRGNYKN

SEQ ID NO: 69 - protein, 20 aa, Saccharomyces cerevisiae
QGGYQQQFNPQGGRGNYKNF

SEQ ID NO: 70 - protein, 20 aa, Saccharomyces cerevisiae
GGYQQQFNPQGGRGNYKNFN

SEQ ID NO: 71 - protein, 20 aa, Saccharomyces cerevisiae
GYQQQFNPQGGRGNYKNFNY

SEQ ID NO: 72 - protein, 20 aa, Saccharomyces cerevisiae
YQQQFNPQGGRGNYKNFNYN

SEQ ID NO: 73 - protein, 20 aa, Saccharomyces cerevisiae
QQQFNPQGGRGNYKNFNYNN

SEQ ID NO: 74 - protein, 20 aa, Saccharomyces cerevisiae
QQFNPQGGRGNYKNFNYNNN

SEQ ID NO: 75 - protein, 20 aa, Saccharomyces cerevisiae
QFNPQGGRGNYKNFNYNNNL

SEQ ID NO: 76 - protein, 20 aa, Saccharomyces cerevisiae
FNPQGGRGNYKNFNYNNNLQ

SEQ ID NO: 77 - protein, 20 aa, Saccharomyces cerevisiae
NPQGGRGNYKNFNYNNNLQG

SEQ ID NO: 78 - protein, 20 aa, Saccharomyces cerevisiae
PQGGRGNYKNFNYNNNLQGY

SEQ ID NO: 79 - protein, 20 aa, Saccharomyces cerevisiae
QGGRGNYKNFNYNNNLQGYQ

SEQ ID NO: 80 - protein, 20 aa, Saccharomyces cerevisiae
GGRGNYKNFNYNNNLQGYQA

SEQ ID NO: 81 - protein, 20 aa, Saccharomyces cerevisiae
GRGNYKNFNYNNNLQGYQAG

SEQ ID NO: 82 - protein, 20 aa, Saccharomyces cerevisiae
RGNYKNFNYNNNLQGYQAGF

SEQ ID NO: 83 - protein, 20 aa, Saccharomyces cerevisiae
GNYKNFNYNNNLQGYQAGFQ

SEQ ID NO: 84 - protein, 20 aa, Saccharomyces cerevisiae
NYKNFNYNNNLQGYQAGFQP

SEQ ID NO: 85 - protein, 20 aa, Saccharomyces cerevisiae
YKNFNYNNNLQGYQAGFQPQ

SEQ ID NO: 86 - protein, 20 aa, Saccharomyces cerevisiae
KNFNYNNNLQGYQAGFQPQS

SEQ ID NO: 87 - protein, 20 aa, Saccharomyces cerevisiae
NFNYNNNLQGYQAGFQPQSQ

SEQ ID NO: 88 - protein, 20 aa, Saccharomyces cerevisiae
FNYNNNLQGYQAGFQPQSQG

SEQ ID NO: 89 - protein, 20 aa, Saccharomyces cerevisiae
NYNNNLQGYQAGFQPQSQGM

SEQ ID NO: 90 - protein, 20 aa, Saccharomyces cerevisiae
YNNNLQGYQAGFQPQSQGMS

SEQ ID NO: 91 - protein, 20 aa, Saccharomyces cerevisiae
NNNLQGYQAGFQPQSQGMSL

SEQ ID NO: 92 - protein, 20 aa, Saccharomyces cerevisiae
NNLQGYQAGFQPQSQGMSLN

SEQ ID NO: 93 - protein, 20 aa, Saccharomyces cerevisiae
NLQGYQAGFQPQSQGMSLND

SEQ ID NO: 94 - protein, 20 aa, Saccharomyces cerevisiae
LQGYQAGFQPQSQGMSLNDF

SEQ ID NO: 95 - protein, 20 aa, Saccharomyces cerevisiae
YQAGFQPQSQGMSLNDFQKQ

SEQ ID NO: 96 - protein, 20 aa, Saccharomyces cerevisiae
GFQPQSQGMSLNDFQKQQKQ

SEQ ID NO: 97 - protein, 20 aa, Saccharomyces cerevisiae
PQSQGMSLNDFQKQQKQAAP

SEQ ID NO: 98 - protein, 20 aa, Saccharomyces cerevisiae
QGMSLNDFQKQQKQAAPKPK

SEQ ID NO: 99 - protein, 20 aa, Saccharomyces cerevisiae
SLNDFQKQQKQAAPKPKKTL

SEQ ID NO: 100 - protein, 20 aa, Saccharomyces cerevisiae
DFQKQQKQAAPKPKKTLKLV

SEQ ID NO: 101 - protein, 20 aa, Saccharomyces cerevisiae
KQQKQAAPKPKKTLKLVSSS

SEQ ID NO: 102 - protein, 20 aa, Saccharomyces cerevisiae
KQAAPKPKKTLKLVSSSGIK

SEQ ID NO: 103 - protein, 20 aa, Saccharomyces cerevisiae
APKPKKTLKLVSSSGIKLAN

SEQ ID NO: 104 - protein, 20 aa, Saccharomyces cerevisiae
PKKTLKLVSSSGIKLANATK

SEQ ID NO: 105 - protein, 20 aa, Saccharomyces cerevisiae
TLKLVSSSGIKLANATKKVG

SEQ ID NO: 106 - protein, 20 aa, Saccharomyces cerevisiae
LVSSSGIKLANATKKVGTKP

SEQ ID NO: 107 - protein, 20 aa, Saccharomyces cerevisiae
SSGIKLANATKKVGTKPAES

SEQ ID NO: 108 - protein, 20 aa, Saccharomyces cerevisiae
IKLANATKKVGTKPAESDKK

SEQ ID NO: 109 - protein, 20 aa, Saccharomyces cerevisiae
ANATKKVGTKPAESDKKEEE

SEQ ID NO: 110 - protein, 20 aa, Saccharomyces cerevisiae
TKKVGTKPAESDKKEEEKSA

SEQ ID NO: 111 - protein, 20 aa, Saccharomyces cerevisiae
VGTKPAESDKKEEEKSAETK

SEQ ID NO: 112 - protein, 20 aa, Saccharomyces cerevisiae
KPAESDKKEEEKSAETKEPT

SEQ ID NO: 113 - protein, 20 aa, Saccharomyces cerevisiae
ESDKKEEEKSAETKEPTKEP

SEQ ID NO: 114 - protein, 20 aa, Saccharomyces cerevisiae
KKEEEKSAETKEPTKEPTKV

SEQ ID NO: 115 - protein, 20 aa, Saccharomyces cerevisiae
EEKSAETKEPTKEPTKVEEP

SEQ ID NO: 116 - protein, 20 aa, Saccharomyces cerevisiae
SAETKEPTKEPTKVEEPVKK

SEQ ID NO: 117 - protein, 20 aa, Saccharomyces cerevisiae
TKEPTKEPTKVEEPVKKEEK

SEQ ID NO: 118 - protein, 20 aa, Saccharomyces cerevisiae
PTKEPTKVEEPVKKEEKPVQ

SEQ ID NO: 119 - protein, 20 aa, Saccharomyces cerevisiae
EPTKVEEPVKKEEKPVQTEE

SEQ ID NO: 120 - protein, 20 aa, Saccharomyces cerevisiae
KVEEPVKKEEKPVQTEEKTE

SEQ ID NO: 121 - protein, 20 aa, Saccharomyces cerevisiae
EPVKKEEKPVQTEEKTEEKS

SEQ ID NO: 122 - protein, 20 aa, Saccharomyces cerevisiae
KKEEKPVQTEEKTEEKSELP

SEQ ID NO: 123 - protein, 20 aa, Saccharomyces cerevisiae
EKPVQTEEKTEEKSELPKVE

SEQ ID NO: 124 - protein, 20 aa, Saccharomyces cerevisiae
VQTEEKTEEKSELPKVEDLK

SEQ ID NO: 125 - protein, 20 aa, Saccharomyces cerevisiae
TEEKSELPKVEDLKISESTH

SEQ ID NO: 126 - protein, 20 aa, Saccharomyces cerevisiae
LPKVEDLKISESTHNTNNAN

SEQ ID NO: 127 - protein, 20 aa, Saccharomyces cerevisiae
LKISESTHNTNNANVTSADA

SEQ ID NO: 128 - protein, 20 aa, Saccharomyces cerevisiae
THNTNNANVTSADALIKEQE

SEQ ID NO: 129 - protein, 20 aa, Saccharomyces cerevisiae
ANVTSADALIKEQEEEVDDE

SEQ ID NO: 130 - protein, 20 aa, Saccharomyces cerevisiae
DALIKEQEEEVDDEVVNDMF

SEQ ID NO: 131 - protein, 20 aa, Saccharomyces cerevisiae
QEEEVDDEVVNDMFGGKDHV

SEQ ID NO: 132 - protein, 20 aa, Saccharomyces cerevisiae
DEVVNDMFGGKDHVSLIFMG

SEQ ID NO: 133 - protein, 20 aa, Saccharomyces cerevisiae
MFGGKDHVSLIFMGHVDAGK

SEQ ID NO: 134 - protein, 20 aa, Candida albicans
SDQQNTQDQLSGAMANASLN

SEQ ID NO: 135 - protein, 20 aa, Candida albicans
QNTQDQLSGAMANASLNGDQ

SEQ ID NO: 136 - protein, 20 aa, Candida albicans
QDQLSGAMANASLNGDQSKQ

SEQ ID NO: 137 - protein, 20 aa, Candida albicans
LSGAMANASLNGDQSKQQQQ

SEQ ID NO: 138 - protein, 20 aa, Candida albicans
AMANASLNGDQSKQQQQQQQ

SEQ ID NO: 139 - protein, 20 aa, Candida albicans
NASLNGDQSKQQQQQQQQQQ

SEQ ID NO: 140 - protein, 20 aa, Candida albicans
LNGDQSKQQQQQQQQQQQNY

SEQ ID NO: 141 - protein, 20 aa, Candida albicans
DQSKQQQQQQQQQQQNYYNP

SEQ ID NO: 142 - protein, 20 aa, Candida albicans
KQQQQQQQQQQQNYYNPNAA

SEQ ID NO: 143 - protein, 20 aa, Candida albicans
QQQQQQQQQNYYNPNAAQSF

SEQ ID NO: 144 - protein, 20 aa, Candida albicans
QQQQQQNYYNPNAAQSFVPQ

SEQ ID NO: 145 - protein, 20 aa, Candida albicans
QQQNYYNPNAAQSFVPQGGY

SEQ ID NO: 146 - protein, 20 aa, Candida albicans
NYYNPNAAQSFVPQGGYQQF

SEQ ID NO: 147 - protein, 20 aa, Candida albicans
NPNAAQSFVPQGGYQQFQQF

SEQ ID NO: 148 - protein, 20 aa, Candida albicans
AAQSFVPQGGYQQFQQFQPQ

SEQ ID NO: 149 - protein, 20 aa, Candida albicans
AQSFVPQGGYQQFQQFQPQQ

SEQ ID NO: 150 - protein, 20 aa, Candida albicans
QSFVPQGGYQQFQQFQPQQQ

SEQ ID NO: 151 - protein, 20 aa, Candida albicans
SFVPQGGYQQFQQFQPQQQQ

SEQ ID NO: 152 - protein, 20 aa, Candida albicans
FVPQGGYQQFQQFQPQQQQQ

SEQ ID NO: 153 - protein, 20 aa, Candida albicans
VPQGGYQQFQQFQPQQQQQQ

SEQ ID NO: 154 - protein, 20 aa, Candida albicans
PQGGYQQFQQFQPQQQQQQY

SEQ ID NO: 155 - protein, 20 aa, Candida albicans
QGGYQQFQQFQPQQQQQQYG

SEQ ID NO: 156 - protein, 20 aa, Candida albicans
GGYQQFQQFQPQQQQQQYGG

SEQ ID NO: 157 - protein, 20 aa, Candida albicans
GYQQFQQFQPQQQQQQYGGY

SEQ ID NO: 158 - protein, 20 aa, Candida albicans
YQQFQQFQPQQQQQQYGGYN

SEQ ID NO: 159 - protein, 20 aa, Candida albicans
QQFQQFQPQQQQQQYGGYNQ

SEQ ID NO: 160 - protein, 20 aa, Candida albicans
QFQQFQPQQQQQQYGGYNQY

SEQ ID NO: 161 - protein, 20 aa, Candida albicans
FQQFQPQQQQQQYGGYNQYN

SEQ ID NO: 162 - protein, 20 aa, Candida albicans
QQFQPQQQQQQYGGYNQYNQ

SEQ ID NO: 163 - protein, 20 aa, Candida albicans
QFQPQQQQQQYGGYNQYNQY

SEQ ID NO: 164 - protein, 20 aa, Candida albicans
FQPQQQQQQYGGYNQYNQYQ

SEQ ID NO: 165 - protein, 20 aa, Candida albicans
QPQQQQQQYGGYNQYNQYQG

SEQ ID NO: 166 - protein, 20 aa, Candida albicans
PQQQQQQYGGYNQYNQYQGG

SEQ ID NO: 167 - protein, 20 aa, Candida albicans
QQQQQQYGGYNQYNQYQGGY

SEQ ID NO: 168 - protein, 20 aa, Candida albicans
QQQQQYGGYNQYNQYQGGYQ

SEQ ID NO: 169 - protein, 20 aa, Candida albicans
QQQQYGGYNQYNQYQGGYQQ

SEQ ID NO: 170 - protein, 20 aa, Candida albicans
QQQYGGYNQYNQYQGGYQQN

SEQ ID NO: 171 - protein, 20 aa, Candida albicans
QQYGGYNQYNQYQGGYQQNY

SEQ ID NO: 172 - protein, 20 aa, Candida albicans
QYGGYNQYNQYQGGYQQNYN

SEQ ID NO: 173 - protein, 20 aa, Candida albicans
YGGYNQYNQYQGGYQQNYNN

SEQ ID NO: 174 - protein, 20 aa, Candida albicans
GGYNQYNQYQGGYQQNYNNR

SEQ ID NO: 175 - protein, 20 aa, Candida albicans
GYNQYNQYQGGYQQNYNNRG

SEQ ID NO: 176 - protein, 20 aa, Candida albicans
YNQYNQYQGGYQQNYNNRGG

SEQ ID NO: 177 - protein, 20 aa, Candida albicans
NQYNQYQGGYQQNYNNRGGY

SEQ ID NO: 178 - protein, 20 aa, Candida albicans
QYNQYQGGYQQNYNNRGGYQ

SEQ ID NO: 179 - protein, 20 aa, Candida albicans
YNQYQGGYQQNYNNRGGYQQ

SEQ ID NO: 180 - protein, 20 aa, Candida albicans
NQYQGGYQQNYNNRGGYQQG

SEQ ID NO: 181 - protein, 20 aa, Candida albicans
QYQGGYQQNYNNRGGYQQGY

SEQ ID NO: 182 - protein, 20 aa, Candida albicans
YQGGYQQNYNNRGGYQQGYN

SEQ ID NO: 183 - protein, 20 aa, Candida albicans
QGGYQQNYNNRGGYQQGYNN

SEQ ID NO: 184 - protein, 20 aa, Candida albicans
GGYQQNYNNRGGYQQGYNNR

SEQ ID NO: 185 - protein, 20 aa, Candida albicans
GYQQNYNNRGGYQQGYNNRG

SEQ ID NO: 186 - protein, 20 aa, Candida albicans
YQQNYNNRGGYQQGYNNRGG

SEQ ID NO: 187 - protein, 20 aa, Candida albicans
QQNYNNRGGYQQGYNNRGGY

SEQ ID NO: 188 - protein, 20 aa, Candida albicans
QNYNNRGGYQQGYNNRGGYQ

SEQ ID NO: 189 - protein, 20 aa, Candida albicans
NYNNRGGYQQGYNNRGGYQQ

SEQ ID NO: 190 - protein, 20 aa, Candida albicans
YNNRGGYQQGYNNRGGYQQN

SEQ ID NO: 191 - protein, 20 aa, Candida albicans
NNRGGYQQGYNNRGGYQQNY

SEQ ID NO: 192 - protein, 20 aa, Candida albicans
NRGGYQQGYNNRGGYQQNYN

SEQ ID NO: 193 - protein, 20 aa, Candida albicans
RGGYQQGYNNRGGYQQNYNN

SEQ ID NO: 194 - protein, 20 aa, Candida albicans
GGYQQGYNNRGGYQQNYNNR

SEQ ID NO: 195 - protein, 20 aa, Candida albicans
GYQQGYNNRGGYQQNYNNRG

SEQ ID NO: 196 - protein, 20 aa, Candida albicans
YQQGYNNRGGYQQNYNNRGG

SEQ ID NO: 197 - protein, 20 aa, Candida albicans
QQGYNNRGGYQQNYNNRGGY

SEQ ID NO: 198 - protein, 20 aa, Candida albicans
QGYNNRGGYQQNYNNRGGYQ

SEQ ID NO: 199 - protein, 20 aa, Candida albicans
GYNNRGGYQQNYNNRGGYQG

SEQ ID NO: 200 - protein, 20 aa, Candida albicans
YNNRGGYQQNYNNRGGYQGY

SEQ ID NO: 201 - protein, 20 aa, Candida albicans
NNRGGYQQNYNNRGGYQGYN

SEQ ID NO: 202 - protein, 20 aa, Candida albicans
NRGGYQQNYNNRGGYQGYNQ

SEQ ID NO: 203 - protein, 20 aa, Candida albicans
RGGYQQNYNNRGGYQGYNQN

SEQ ID NO: 204 - protein, 20 aa, Candida albicans
GGYQQNYNNRGGYQGYNQNQ

SEQ ID NO: 205 - protein, 20 aa, Candida albicans
GYQQNYNNRGGYQGYNQNQQ

SEQ ID NO: 206 - protein, 20 aa, Candida albicans
YQQNYNNRGGYQGYNQNQQY

SEQ ID NO: 207 - protein, 20 aa, Candida albicans
QQNYNNRGGYQGYNQNQQYG

SEQ ID NO: 208 - protein, 20 aa, Candida albicans
QNYNNRGGYQGYNQNQQYGG

SEQ ID NO: 209 - protein, 20 aa, Candida albicans
NYNNRGGYQGYNQNQQYGGY

SEQ ID NO: 210 - protein, 20 aa, Candida albicans
YNNRGGYQGYNQNQQYGGYQ

SEQ ID NO: 211 - protein, 20 aa, Candida albicans
NNRGGYQGYNQNQQYGGYQQ

SEQ ID NO: 212 - protein, 20 aa, Candida albicans
NRGGYQGYNQNQQYGGYQQY

SEQ ID NO: 213 - protein, 20 aa, Candida albicans
RGGYQGYNQNQQYGGYQQYN

SEQ ID NO: 214 - protein, 20 aa, Candida albicans
GGYQGYNQNQQYGGYQQYNS

SEQ ID NO: 215 - protein, 20 aa, Candida albicans
GYQGYNQNQQYGGYQQYNSQ

SEQ ID NO: 216 - protein, 20 aa, Candida albicans
YQGYNQNQQYGGYQQYNSQP

SEQ ID NO: 217 - protein, 20 aa, Candida albicans
QGYNQNQQYGGYQQYNSQPQ

SEQ ID NO: 218 - protein, 20 aa, Candida albicans
GYNQNQQYGGYQQYNSQPQQ

SEQ ID NO: 219 - protein, 20 aa, Candida albicans
YNQNQQYGGYQQYNSQPQQQ

SEQ ID NO: 220 - protein, 20 aa, Candida albicans
NQNQQYGGYQQYNSQPQQQQ

SEQ ID NO: 221 - protein, 20 aa, Candida albicans
QNQQYGGYQQYNSQPQQQQQ

SEQ ID NO: 222 - protein, 20 aa, Candida albicans
NQQYGGYQQYNSQPQQQQQQ

SEQ ID NO: 223 - protein, 20 aa, Candida albicans
QQYGGYQQYNSQPQQQQQQQ

SEQ ID NO: 224 - protein, 20 aa, Candida albicans
QYGGYQQYNSQPQQQQQQQS

SEQ ID NO: 225 - protein, 20 aa, Candida albicans
YGGYQQYNSQPQQQQQQQSQ

SEQ ID NO: 226 - protein, 20 aa, Candida albicans
GGYQQYNSQPQQQQQQQSQG

SEQ ID NO: 227 - protein, 20 aa, Candida albicans
GYQQYNSQPQQQQQQQSQGM

SEQ ID NO: 228 - protein, 20 aa, Candida albicans
YQQYNSQPQQQQQQQSQGMS

SEQ ID NO: 229 - protein, 20 aa, Candida albicans
QQYNSQPQQQQQQQSQGMSL

SEQ ID NO: 230 - protein, 20 aa, Candida albicans
NSQPQQQQQQQSQGMSLADF

SEQ ID NO: 231 - protein, 20 aa, Candida albicans
PQQQQQQQSQGMSLADFQKQ

SEQ ID NO: 232 - protein, 20 aa, Candida albicans
QQQQQSQGMSLADFQKQKTE

SEQ ID NO: 233 - protein, 20 aa, Candida albicans
QQSQGMSLADFQKQKTEQQA

SEQ ID NO: 234 - protein, 20 aa, Candida albicans
QGMSLADFQKQKTEQQASLN

SEQ ID NO: 235 - protein, 20 aa, Candida albicans
SLADFQKQKTEQQASLNKPA

SEQ ID NO: 236 - protein, 20 aa, Candida albicans
DFQKQKTEQQASLNKPAVKK

SEQ ID NO: 237 - protein, 20 aa, Candida albicans
KQKTEQQASLNKPAVKKTLK

SEQ ID NO: 238 - protein, 20 aa, Candida albicans
TEQQASLNKPAVKKTLKLAG

SEQ ID NO: 239 - protein, 20 aa, Candida albicans
QASLNKPAVKKTLKLAGSSG

SEQ ID NO: 240 - protein, 20 aa, Candida albicans
LNKPAVKKTLKLAGSSGIKL

SEQ ID NO: 241 - protein, 20 aa, Candida albicans
PAVKKTLKLAGSSGIKLANA

SEQ ID NO: 242 - protein, 20 aa, Candida albicans
KKTLKLAGSSGIKLANATKK

SEQ ID NO: 243 - protein, 20 aa, Candida albicans
LKLAGSSGIKLANATKKVDT

SEQ ID NO: 244 - protein, 20 aa, Candida albicans
AGSSGIKLANATKKVDTTSK

SEQ ID NO: 245 - protein, 20 aa, Candida albicans
SGIKLANATKKVDTTSKPQS

SEQ ID NO: 246 - protein, 20 aa, Candida albicans
KLANATKKVDTTSKPQSKES

SEQ ID NO: 247 - protein, 20 aa, Candida albicans
NATKKVDTTSKPQSKESSPA

SEQ ID NO: 248 - protein, 20 aa, Candida albicans
KKVDTTSKPQSKESSPAPAP

SEQ ID NO: 249 - protein, 20 aa, Candida albicans
DTTSKPQSKESSPAPAPAPA

SEQ ID NO: 250 - protein, 20 aa, Candida albicans
SKPQSKESSPAPAPAPAAFA

SEQ ID NO: 251 - protein, 20 aa, Candida albicans
QSKESSPAPAPAPAAFASQE

SEQ ID NO: 252 - protein, 20 aa, Candida albicans
ESSPAPAPAPAAFASQEEKK

SEQ ID NO: 253 - protein, 20 aa, Candida albicans
PAPAPAPAAFASQEEKKEEK

SEQ ID NO: 254 - protein, 20 aa, Candida albicans
APAPAAFASQEEKKEEKEAA

SEQ ID NO: 255 - protein, 20 aa, Candida albicans
FASQEEKKEEKEAAAATPET

SEQ ID NO: 256 - protein, 20 aa, Candida albicans
KKEEKEAAAATPETKKETSA

SEQ ID NO: 257 - protein, 20 aa, Candida albicans
AAAATPETKKETSAPAETKK

SEQ ID NO: 258 - protein, 20 aa, Candida albicans
ETKKETSAPAETKKEATPTP

SEQ ID NO: 259 - protein, 20 aa, Candida albicans
SAPAETKKEATPTPAAKKES

SEQ ID NO: 260 - protein, 20 aa, Candida albicans
KKEATPTPAAKKESTPTPAT

SEQ ID NO: 261 - protein, 20 aa, Candida albicans
TPAAKKESTPTPATATKKES

SEQ ID NO: 262 - protein, 20 aa, Candida albicans
ESTPTPATATKKESTPVSNS

SEQ ID NO: 263 - protein, 20 aa, Candida albicans
ATATKKESTPVSNSASVATA

SEQ ID NO: 264 - protein, 7 aa, Artificial Sequence
  Description of Artificial Sequence Synthetic peptide
XGGYQXN

SEQ ID NO: 265 - protein, 8 aa, Artificial Sequence
  Description of Artificial Sequence Synthetic peptide
RGGYQXNN

SEQ ID NO: 266 - protein, 7 aa, Artificial Sequence
  Description of Artificial Sequence Synthetic 7x His tag
HHHHHHH

SEQ ID NO: 267 - protein, 6 aa, Artificial Sequence
  Description of Artificial Sequence Synthetic 6x His tag
HHHHHH